(19) United States
(12) Patent Application Publication
(10) Pub. No.: US 2014/0358150 A1
(43) Pub. Date: Dec. 4, 2014

(54) SURGICAL DISTRACTION DEVICE WITH EXTERNAL ACTIVATION

(71) Applicant: Children's National Medical Center, Washington, DC (US)

(72) Inventors: Roger E. Kaufman, Washington, DC (US); Anthony D. Sandler, Washington, DC (US)

(73) Assignee: Children's National Medical Center, Washington, DC (US)

(21) Appl. No.: 14/289,616

(22) Filed: May 28, 2014

Related U.S. Application Data
(60) Provisional application No. 61/828,656, filed on May 29, 2013.

(51) Int. Cl. A61B 17/02

(52) U.S. Cl. CPC A61B 17/025; A61B 2017/0256
USPC 606/90

(57) ABSTRACT

A surgical distraction apparatus is disclosed. The surgical distraction apparatus includes a tissue distraction device adapted for implantation, the tissue distraction device including a plurality of distraction members, one or more actuators adapted for implantation and operatively coupled to the tissue distraction device, the one or more actuators being configured to drive the tissue distraction device and adjust the alignment of the plurality of distraction members, and an activator adapted to deliver one or more pulses to at least one actuator in the one or more actuators.

### RELATED APPLICATION DATA

This application claims priority to U.S. Provisional Application No. 61/828,656, filed May 29, 2013, the disclosure of which is hereby incorporated by reference in its entirety.

### BACKGROUND

Distraction systems are utilized for altering body configuration and skeletal malformation. Progressive distraction is critical to many craniofacial, orthopedic and surgical interventions, such as for scoliosis, which is a three-dimensional deformation of the spine. Idiopathic scoliosis is typically corrected by the surgical insertion of rods attached to two or more vertebrae. During traditional surgery, high forces are applied, distracting the vertebrae from their initial position so as to partially or fully correct the scoliosis. This sudden mechanical stretching of the tissues is traumatic and painful and results in high stresses in the implanted rod system.

As another illustrative example, pectus excavatum is the most common congenital deformity of the anterior chest wall in which the sternum and costochondral cartilages are abnormally formed. Previously, rigid metal bars were typically inserted to distract the chest wall and correct the deformity in a single operation.

Craniosynostosis is a condition in which the sutures of an infant's skull are prematurely closed. Currently these sutures are surgically opened by one or more operations in which the bones of the skull are removed or reshaped.

Common to most prior distraction systems is a mechanism based on the physical manipulation of the system by the surgeon. Such systems generally necessitate open access to the implanted device to alter its configuration. Frequently these systems are applied externally or internally with external exposure of the unit and are fraught with an increased morbidity and risk of infection. Many such systems are limited by the inability to change the configuration of the implanted hardware as the patient's habitus changes. Non-static systems are even more germane to changes with growth observed in a pediatric population.

Current techniques for surgical correction of malformations often require sudden, radical stretching and correction of the tissue configuration in each operation so as to minimize the number of invasive surgical procedures required. In some cases there is a need for multiple operations, for example, to allow for growth or for swelling to subside. Often this entails severe postoperative trauma and pain. It also places a strain on resources for both the patient and society.

A small number of surgical distraction devices have been developed which can be externally activated once the incisions are closed.

Typically these systems depend on the ability to place a toroidal magnet around the limb for the purpose of moving the internal elements of the device as in a conventional electric motor with a rotor and a stator. Due to these spatial constraints such systems are limited in application to use in extremities, such as the femur. They can't be employed within the chest or abdomen, for example.

Other devices employ implanted electric motors or components containing rare earth elements that are potentially toxic within the human body.

Several systems have been developed in which an implanted actuator lengthens or shortens a rod or cable. Tissue distraction is intended to occur due to axial compression or tension forces in the rod or cable transmitted in-line to the tissues.

Treatment of scoliotic diseases is typically accomplished by spine fixation using specially designed pedicle screws or other devices. These are attached to suitable points on several of the vertebral bodies and to rigid spinal rods by means of a variety of specialized connection devices. Many of these fixation devices provide adjustable degrees of freedom that allow the surgeon some intra-operative leeway to adjust the positions of the vertebrae relative to one another in the coronal, sagittal, and transverse planes. However a common characteristic of most such spinal fixation systems is that postoperatively there is no possibility of making minor adjustments to the geometry of the fixation system to correct for growth or to accommodate changes in the anatomy due to the presence of the fixation system over a period of time.

During surgery, the fixation system is generally pre-tensioned to urge the spine into a more desired configuration. Unfortunately, once the vertebrae have relaxed in the desired direction a state of equilibrium is attained in which there are no longer any significant restoring forces to further coerce the tissues and further correct the deformity.

In fact, the presence of the implanted rigid fixation system can eventually begin to have an adverse effect, constraining growth and necessitating further operations to shift the rod connections to new spatial locations where they can again provide the desired corrective loads on the spinal column.

“Dynamic” or semi-constrained vertebral couplings have been used in the past, for instance with Harrington rods. Using screws in slots or cylindric joints sliding between stops, these dynamic couplings allow axial or angular motions to compensate for subsidence or settling.

Telescoping rod systems have also been developed. Most of these are extended by surgical intervention with accompanying trauma and risks.

Kiester, U.S. Pat. No. 7,955,357, describes an externally adjustable expandable rod system for the treatment of scoliosis. This utilizes an implanted Nitinol muscle wire ratchet actuator to lengthen an implanted rod. Electrically heating and cooling the Nitinol wire via an implanted power pickup causes the Nitinol wire to alternately stretch and shrink. This motion incrementally rotates a screw system which telescopically lengthens the rod over a period of time. In this way, the rod is loaded in compression thereby gradually forcing apart the vertebrae attached to the two ends of the rod. Scoliosis correction with this device is primarily obtained by axial (compressive) forces in the rod rather than lateral bending (side to side) forces on the vertebrae as is done in the present system.

Nuss, U.S. Pat. No. 6,024,759, describes a minimally invasive treatment for pectus excavatum in which one or more bowed steel bars are inserted under the deformed sternum with their convexity facing posteriorly. Subsequently, in the same operation, the bars are rotated 180 degrees so that the ends of the bars follow the curvature of the chest and the deformed sternum is forced outwards towards its final position in a single operation. Unfortunately, this sudden surgical correction of the deformity is generally accompanied by severe postoperative trauma and pain.

Distraction osteogenesis is the process of gradually correcting deformities by stretching bones and tissues such as the long bones of the leg or craniofacial bones. A variety of devices have been developed for this purpose, including external fixators, internal nails, and motorized implants.

Intramedullary nails driven by electric motors and control systems have been designed for elongating long bones separated by an osteotomy. Such systems require implantation of electric motors with potentially toxic rare earth magnetic materials and are tailored to use in the long bones of the extremities.

Stepping motors, pawls, and ratchet devices have long been applied in non-medical applications such as clock escapements, typewriters, printers, odometers, toys, wrenches, motion picture projectors, rotary telephone switchboards, production machinery and the like.

Magnetically activated clock escapements and single cycle clutches have been employed to incrementally release a pre-tensioned spring or a shaft driven by some other power source. Solenoid driven ratchet stepping motors have also been used but such devices require a source of electricity to drive the ratchet. Further, the solenoid magnet that is built into the assembly depends on precise alignment and a small controlled gap to insure proper functioning of the device.

Similarly, electrical stepping motors generally utilize a rotor and a stator. They incorporate multiple pole pieces and windings and require an electrical power source to sequentially energize the windings. They generally depend on a small carefully controlled air gap between the rotor and the stator so as to reduce the length of the flux lines between the fixed and moving pole pieces. A rotating magnetic field generated within the windings causes the rotor to index relative to the stator. To get a significant amount of torque, either the rotor or the stator must be energized or else a permanent magnet is employed. Strong permanent magnets generally are highly toxic due to their rare earth element composition.

### BRIEF SUMMARY

A system and apparatus is described that provides a distraction system that can be externally adjusted and manipulated after the incisions are closed and without necessitating further surgical intervention. The ability of the present system to gradually make the transition from an initial to the planned final configuration can radically change the feasibility and efficacy of surgical distraction. In many cases it can greatly speed recovery while also reducing pain, trauma, number of surgical interventions required, and costs both to the patient and to society.

The present system provides a distraction apparatus with a final configuration that can be preoperatively planned so as to produce the intended corrected geometry, optionally over an extended period of time but with an initial configuration that can closely conform to the preoperative geometry of the patient.

The present system provides a distraction apparatus that can be reconfigured through a minimally invasive incision.

The present system also provides a minimally invasive distraction apparatus that can synchronously provide desired amounts of progressive correction at multiple points using a single externally controlled actuator. In many cases, the proportional amounts of displacement to be applied at each point during each treatment can be preoperatively determined.

The present system describes a spinal fixation apparatus that can be adjusted postoperatively over a period of time, requiring a minimum of surgical intervention and minimizing the associated trauma, such as the trauma traditionally associated with correction of Idiopathic scoliosis.

Similarly, the present distraction apparatus describes a system that can slowly correct various other malformations or deformities thereby allowing the tissues to relax and equilibrate over time. In this way, use of the present apparatus can avoid much postoperative pain and discomfort, such as that normally associated with treatment of pectus excavatum, pectus carinatum, or craniosynostosis.

The present system is directed to a family of cable coupled surgical distraction apparatuses and systems that use an externally controlled implanted incremental actuator. These apparatuses and systems allow slow progressive alteration of the patient's physical configuration via external control such as by an externally controlled electromagnetic field or physical pressure on the skin. The present system aids in minimally invasive surgical procedures.

The system includes one or more implantable ratchet-driven actuators, an implantable tissue distraction means coupled to and driven by said actuators, and an external actuation means for incrementally activating said actuators by delivering a sequence of magnetic or mechanical pulses. Other forms of actuator and actuation means can also be employed. Such means can utilize electric motors controlled by means of wireless telemetry, induction coils, or other modalities or means such as manually operated levers or threaded devices.

The system also includes surgical distraction apparatuses with segmented cable-tensioned distraction members. These members are so arranged and disposed as to provide a multiplicity of lateral (bending) forces at right angles to the members and at multiple locations and not just axial forces along the members. These combined forces gradually urge the distracted tissues in planned directions as the cable tension is increased. Such cable-tensioned distraction members can include segmented rods or bars adapted to insertion through relatively small surgical openings. These segmented structures can be tensioned into their final rigid configuration. Such tensioning can be done progressively over a period of time using the externally controlled actuator means of the present system.

Such tensioning can also be achieved surgically using an implanted adjusting means rather than with an externally controlled tensioning actuator. Tensioning can be done either prior to closing the initial incision or progressively over a period of time using a minimal incision to gain access to the tensioning means.

A compliant spring means can be incorporated in series with the cable system. This spring means can be designed to protect against accidental overloads of the mechanism or of the tissues being distracted. It can also be designed so as to provide a consistent or preferred level of tension in the cables independent of perturbations caused by relative motions of the bones or other structures. Flexural properties can be built into the system's attachments or anchors, into selected links, or into the tensioning cables themselves.

The present system is particularly suited to minimally invasive surgical procedures, since many of the components are adapted to insertion through relatively small surgical openings and then can be tensioned into their final configuration while working through the same surgical incision proximal to the actuator itself.

The implanted actuators can be powered by a pulsed magnet system external to the patient, optionally directed by computer control.

Post-surgery, the implanted actuators can be externally activated in a controlled fashion without further surgical intervention and without any penetration of the skin by wires or other devices.

The magnetically controlled actuator of the present system uses neither implanted magnets nor implanted power sources or heating elements. It also does not depend on having a small, carefully aligned air gap or the ability to completely surround the actuator motor with a rotating magnetic field. Thus, the present distraction system can be used on portions of the anatomy such as the chest or abdomen where the actuator is only proximal to one surface of the body.

The system does not require implantation of any rare earth elements, batteries, electric motors, or other toxic substances. The implanted parts are purely passive mechanical components. Additionally, many variations of the system and apparatus described can be utilized. The ratchet actuators can be driven by mechanical pressure applied to portions of the actuator through the tissues of the skin. Actuation of the cable-tensioned distraction system can be achieved surgically through a minimally invasive incision. Actuators of the present system can be configured to produce either rotary or linear incremental motions.

These rotary or linear motions from the actuator are mechanically coupled to what will herein be referred to as “end effector” members that in turn push or pull on the actual tissues of the patient thereby performing the actual distraction.

A number of such novel coupling means can be used together with a number of such novel end effector distraction devices. For example, the actuators can be used to stiffen a segmented cable-tightened system of link elements. In the slack, unstressed configuration, these links can be threaded through contorted anatomical regions so as to conform to the basically uncorrected pre-surgical deformity.

The geometry of the contact points or joints between the link elements is arranged so that as the links are progressively pulled into the stiffened configuration by means of the aforementioned actuator, one or more points on the various links applies pressure to the tissues urging them into the desired final distracted configuration.

Construction of the joints between the tensioned segments can be adjusted to constrain the direction of flexing at that joint and to allow preferred combinations of distraction forces on the tissues or bones being distracted. These can consist of a mix of lateral forces (perpendicular to the segments) and axial forces (along the segments). The directions and magnitudes of these forces can vary from segment to segment and also over time with the progression of the treatment as the system is incrementally tensioned postoperatively.

The present system teaches both planar and non-coplanar constructions for the distraction apparatus. Non-coplanar distraction displacements, forces, and torques can be produced using either the planar or non-coplanar apparatus constructions taught herein.

The present system also teaches how the geometry of the joints between the tensioned segments can be tailored so that the joints between adjacent segments become stiffer and more rigid as the tensioning cable is shortened. In effect, the individual links behave more like a single member better able to resist transverse and bending loads as the treatment progresses.

The final end effector action between the links and the tissues can be accomplished by means of a number of devices, such as pedicle screws, sutures, wires, adjustable rod mounts, and so forth; by direct contact pressure between various bearing surfaces on the links and the tissues; or by other coupling means allowing desired directions of freedom and constraint. Elastically compliant elements can be incorporated into some end effectors to control the force levels at that interface between the tissues and the distraction device.

One or more of said end effector coupling means can be engaged to said actuator in such a way as to simultaneously apply corrective forces or pressures to several areas of the patient's anatomy as needed.

These can include original end effector coupling means tailored to the needs of a particular patient or surgical operation or such well-known means as pedicle screws, sutures, cables, rods, ball joints, compliant couplings, point, line, or surface contacts, and so forth. Depending on the particular application of the system a plurality of such end effector couplings can be used at the same time.

The present system also teaches construction of some new end effector pedicle attachment devices for transferring forces between the jointed distraction rod members and the vertebrae in the treatment of scoliosis.

The actuator of the present system can be used to drive one or more screws and nuts or turnbuckle-type devices to pull together or push apart bony structures such as vertebrae, skull sutures, or for other tissue distraction purposes. Again, the final attachment of these nuts or turnbuckles to the tissues can be attained by various devices.

Coupling between the rotary actuator and these turnbuckles, cables, or other devices can be accomplished by a variety of mechanisms providing the necessary amount of torsional or rotational stiffness. However said coupling can also be so configured as to allow flexibility and out of plane motions where desired or when the actuator is not proximal to the distraction end effector. For instance, rigid rods or shafts can be used but there can also be flexible shafts, telescoping prismatic joints, bellows, bell cranks, universal joints, or other equivalent means used to provide needed flexibility and room for growth or to accommodate the internal motions of the anatomy while still providing and constraining the desired distraction.

A single rotary actuator can drive multiple distractor end effectors. Distraction performance can be tailored to attain a differential action at various points along the mechanism by a variety of means. For example, linkages or different screw pitches can be employed at different points in the system thereby tightening some points while loosening others.

Some of these turnbuckles or nuts can employ a variety of left or right-hand screw pitches providing a differential action as needed so that a unidirectional rotation of the actuator can cause various forms of distraction at different points in the body. For instance in the case of the spine, some points can be spread apart while others were drawn together.

The actuator can be used to drive a flexible rotary coupling system such as a flexible shaft, telescoping prismatic joints, bellows, or universal joints, thereby conveying the rotary motion to remote sites within the body where it can be needed for tissue distraction or other purposes.

It will be appreciated that the actuator can have single or double-ended shafts as needed. Further, it can be coupled via gear trains, levers, cables, push-pull shafts, power screws, or other devices so as to obtain increased mechanical advantage if needed.

It will be further noted that a variety of end effector means can be used to couple the tissue distraction motions of the actuator-driven components of the present system to the anatomy of the patient so as to most effectively utilize the distraction system in a particular application.

For example, in correction of pectus excavatum, a multi-link cable-tensioned hinged assembly of rods or bars can be passed beneath the sternum from one side of a patient to the other. This initial operation can be performed in a minimally invasive manner using tapes or cords to pull the loosely coupled link members beneath the tissues following the curvature of the uncorrected anatomy. A protective flexible sleeve can be used as a passageway to assist in guiding these components into position and to provide protection to the tissues, both during the initial operation and optionally for the duration of the progressive treatment. One or more incremental or other rotary actuators or terminal fixation devices can then be coupled to the ends of this cable system and sutured beneath the skin on the patient's sides. Initially, the link assembly can conform to the patient's deformity and can be relatively unstressed.

Postoperatively, over a period of time, the actuators in the present system can be externally activated, incrementally tensioning the link system without further surgical interventions. The geometry of the links and their coupling joints can be so configured as to gradually apply necessary corrective distraction forces to the sternum as the link assembly becomes stiffer. In this situation, the end effectors conveying the distraction forces to the tissues can be the contact surfaces of the actual links themselves or a sleeve or other surface between the tissues and said links.

Controlling the number of incremental activations of the actuators can provide a measure of the amount of correction being delivered during any particular postsurgical treatment and, indirectly, the change in distraction forces that can be expected as a result. Over a period of weeks or months these forces can slowly urge the tissues into the desired final form without the sudden pain and discomfort of the traditional open surgical procedure.

As another typical application of the present system, in surgical correction of scoliosis a multi-link cable-tensioned hinged assembly of rods can be tunneled or implanted alongside the spine and attached to two or more selected vertebrae using pedicle screws or other devices as described earlier. Initially, the slack joints between these rod segments can allow them to somewhat conform to the spine's initial deformity.

An actuator of the present system can be attached to the cables at one distal end of the cables. Over a period of time the cables can be tensioned. Contact forces between adjacent links can cause the links to move towards a final predetermined configuration. The geometry of the various joints between the links and the shape of the links themselves can determine the final three-dimensional shape of the distraction device. The end effectors acting on the various vertebrae (pedicle screws, compliant or sliding connectors, or other devices) can apply relatively gentle loads on the spine over a period of time, thereby urging the ligamentum flavum to stretch so as to alleviate the scoliosis.

Selected vertebral attachments can be coupled to jointed links by slack motion couplings of the present system utilizing slots, cams, or pivots together with mechanical limit stops. These limit stops can be positioned so as to allow some parts of the distraction system to move freely in certain directions when treatment begins and only begin applying distraction force to the connected structures at a later point in the progressive treatment protocol. In another illustrative example, they can also be configured to provide initial corrective forces and displacements at certain locations that taper off and diminish later in the progressive treatment while other portions of the distractor apparatus might come into engagement or continue to apply loads at other locations.

In some situations a pair of cable-tensioned apparatuses can be implanted on either side of the spine to apply more control and a higher corrective force. In this case, actuators of the present system can be offset from one another to allow each cable system to be independently controlled and tensioned.

Cross members coupling selected jointed members of the two rod assemblies can be provided with needed pivots or other joints allowing needed flexibility in desired planes and stiffness and constraint in other directions. These cross members can also provide fixation supports for pedicle screws and similar hardware.

A variety of other mechanisms can be driven by the incremental actuator system and used to provide the distraction forces. For instance: single or multiple cables can be employed; the link members can be hinged together or keyed to interlock by the shape of their contacting surfaces; a single flexible spline member with controllable stiffness can be used in place of the multiple links; the links can be bent or have varying cross-sections; there can be intermediate guides or attachments; and so forth.

Further, a variety of motor devices with suitable gear reductions and control systems can possibly be utilized in place of the incremental actuator to activate the mechanical distraction system. When utilizing such motors, power and control devices can be implanted along with the motor actuator. Inductive coupling or other means can be used to power the implanted motor to avoid the use of skin penetrating wires. Additionally, Nitinol or electric motor drive systems can be used in place of the ratchet actuators described herein.

### BRIEF DESCRIPTION OF THE DRAWINGS

FIG. 1 is a schematic conceptual overview showing the main elements of the system as it can be configured for treating scoliosis.

FIG. 2 illustrates a distraction system for treating pectus excavatum according to an exemplary embodiment.

FIG. 3 illustrates the system's segmented cable-tensioned distraction members according to an exemplary embodiment.

FIG. 4 is an illustration of how the present system guides the distraction members into alignment as the cables bring the mating surfaces into contact.

FIGS. 5-6 illustrate how the tubular links of the present system can be configured with one or more cables and specialized joint geometry to better control the relative motion between the distraction members.

FIGS. 7-8 illustrate how substantially bar-shaped links of the present system can be configured to “nest” and come into rigid alignment in a two-dimensional application.

FIG. 9 illustrates an overview of the geometry needed to understand the principles underlying operation of the joints of the present system.

FIG. 10 is a two-dimensional example illustrating how joint geometry of the present system can force proper self-assembly by restricting possible translational freedoms between links.

FIGS. 11-13 show how the instantaneous joint configuration of the same two links insures proper self-assembly by forcing the links into rotational alignment.

FIG. 13 details the overall rotational freedom and constraint provided by joints of the present system.

FIG. 14 illustrates a simplified diagram of the forces acting at a typical joint of the present system as the cables are tightened.

FIG. 15 illustrates the polygon of forces at the joint in FIG. 9. The cable tension is in equilibrium with the forces at the contact patches between the links

FIGS. 16-18 illustrate how the principles taught in the FIGS. 7-15 can be generalized and extended to three-dimensional alignment variations.

FIG. 17 illustrates how the links of the present system can be inserted and threaded along a curved guide cable prior to tensioning.

FIG. 18 illustrates how the links of the present system can be straight or curved and how asymmetrical joint geometry can coerce adjacent links into alignment as the joints are mated together.

FIG. 19 gives a schematic overview of the present system.

FIG. 20 illustrates an external pulse generator used to incrementally drive the implanted distraction actuator system according to an exemplary embodiment.

FIG. 21 outlines a control computer that can be used to operate the present system.

FIG. 22 is a block diagram of key elements of the power switching and control system according to an exemplary embodiment.

FIG. 23 is an exterior view of an actuator of the present system according to an exemplary embodiment.

FIG. 24 illustrates an exemplary embodiment of the incremental actuation surgical motor of the present system.

FIG. 25 is an exploded view of the wrap spring actuator of FIG. 24.

FIG. 26 illustrates an exemplary embodiment of the present ratchet actuator system as it can be arranged for treatment of pectus excavatum and which uses a multiplicity of pawls to produce micro-stepping despite limited angular rotation of the input lever.

FIG. 27 illustrates the FIG. 26 device mounted in a case with typical suture attachments, a coupling system for connecting the actuator to tension cables, and an adjusting provision for positioning the actuator relative to a reaction member against which the cables are tensioned.

FIG. 28 illustrates a manually actuated reversible ratchet actuator using a wrap spring clutch which can be activated by external pressure on the skin of the patient.

FIG. 29 illustrates a dual ended system of the actuators according to an exemplary embodiment.

FIG. 30 illustrates how gearboxes can be employed to drive end effectors in other planes, at different speeds, at right angles to the primary actuator shaft, or to increase the available torque.

FIGS. 31 and 32 illustrate how multiple distractors of the present system can be staggered for use in treatment of scoliosis.

FIG. 33 illustrates how the distraction apparatus of the present system can be coupled to vertebrae.

FIG. 34 illustrates a pedicle attachment of the present system allowing angular compensation during the course of treatment.

FIG. 35 illustrates how the pedicle attachment system of the present system can allow selective limited axial or angular motion with respect to the distractor links during the course of treatment.

FIG. 36 details a split pedicle attachment system of the present system.

FIG. 37 illustrates how limit stops of the present system can be incorporated to further control the timing and the distraction applied at different points during the course of treatment.

FIG. 38 illustrates a dual distractor system and how limit stops of the present system can be incorporated to control when during treatment a particular distractor force becomes active.

FIGS. 39 and 40 show a collar illustrating how a limit stop of the present system can control when during treatment a particular axial distractor force becomes active.

FIG. 41 illustrates the present system using a turnbuckle end effector distraction device coupled to the actuator by means of a flexible shaft. Right or left-handed screw threads can be chosen for the turnbuckle's threaded members allowing the end effector distractor to draw together or separate as needed, for a given direction of rotation of the driving actuator.

FIG. 42 illustrates a turnbuckle end effector distractor of the present system that can be applied to craniosynostosis or other surgical procedures requiring separating or drawing together bony structures, and in which differential screw threads can be chosen to provide high forces and fine distraction adjustments.

FIG. 43 illustrates an exemplary embodiment of the present system in which telescoping or universal joints are chained together to permit the actuator to be remotely mounted relative to the distractor end effector and illustrates the variations allowed by the interchanging modular components of the system.

FIG. 44 illustrates an exemplary embodiment of the present system in which a tension cable is employed as the terminal end effector distraction device.

FIG. 45 illustrates the present system employing a turnbuckle actuated hinged distractor according to an exemplary embodiment.

FIG. 46 illustrates the present system wherein multiple distraction devices are serially coupled together by flexible shafts or other devices and driven by a single implanted actuator according to an exemplary embodiment.

FIG. 47 is a chart showing possible variations of the design of the implantable surgical distraction apparatus and conditions which can be treated using the apparatus.

### DETAILED DESCRIPTION

The present system and apparatus is directed to an implantable surgical distraction device suited to application for correction of a wide variety of anatomical malformations. The device is particularly directed towards use in protracted treatments extending over long periods where the degree of distraction can benefit by being incrementally altered long after the original surgical operation has ended. Scoliosis and pectus excavatum are typical examples of such surgeries where a capability for making post-surgical adjustments can be beneficial.

Implantable elements of the current system are particularly suited to insertion by minimally invasive surgical procedures. In many cases they can be threaded into the body through relatively small incisions, pulled or pushed through like beads on a string and then erected in place by tensioning a device at the terminal end of the assembly.

Elements of the present system are modular in concept and can be employed in various combinations as needed to meet the surgical situation at hand. Further, some of these components can be custom-shaped to meet the size and needs of a particular patient. Certain components are adapted to receive custom attachments and ancillary hardware for purposes such as anchoring sutures or fixating pieces to various bony structures such as pedicles of the vertebral bodies or ribs.

The current system is adapted to progressively changing its spatial arrangement from an initial configuration somewhat following the contours of the patient's malformation at the time of surgery to a final corrected configuration. As the distraction device changes shape, the change of shape is communicated to the patient's tissues via “end effectors” which press on selected portions of the anatomy and cause the tissues to relax towards a preferred corrected arrangement.

This change of geometry can be controlled by means external to the patient without necessitating further surgery for each adjustment. For instance, in many cases it can be done during a series of office visit treatments.

The features which correspond to the numerals in the figures in an exemplary embodiment are listed below and are discussed in greater detail with respect to each figure.
10—Surgical motor
20—Typical distraction device and coupling mechanism
21—Before treatment
22—After treatment
23—Transverse section of abdomen
30—External actuator drive means
40—Control system
50—Link segment
55—End Effectors
60—Tensioning cables
70—Contact between members
72—Contact surfaces between members
74—Cable passageways
76—Hinge axis
80—Cylindrical tubular link assembly
90—Cylindrical tubular link body
100 —Hinging joint members prior to tensioning
110 —Hinging joint members post tensioning
120 —Female joint half
130—Male joint half
135—Sliding cylindric surfaces
140—Rigidly aligned nesting joints
150—Contact surfaces between members
160—Links brought into alignment by joint geometry
170—Female keyed joint half
180—Male keyed joint half
190—Keyed and fully mated joint
200—Curved link
210—Straight link
220—Mated pair of links
230—Tensioning cable passages
240—Contact as joints start to close
250—Fully closed joint
260—Rounded nose
262—Rounded trailing edge
270—Wedge surfaces
280—Parallel locking surfaces
290—Final seating surfaces
300—Reference link
310—Link moving with respect to reference link
320—Contact patch at O₁
330—Contact patch at O₂
340—Common normal at contact patch O₁
350—Common normal at contact patch O₂
360—Common tangent at contact patch O₁
370—Common tangent at contact patch O₂
380—Field of translational restraint due to contact at O₁
390—Field of translational freedom due to contact at O₁
400—Field of translational restraint due to contact at O₂
410—Field of translational freedom due to contact at O₂
420—Overall field of translational freedom in joint
430—Fields of Clockwise Rotational Freedom allowed by contact at O₁ (Solid curved arrows)
440—Fields of Counter-Clockwise Rotational Freedom allowed by contact at O₁ (Solid curved
arrows)
450—Fields of Clockwise Rotational constraint allowed by contact at O₁ (Dashed curved arrows)
460—Fields of Counter-Clockwise Rotational constraint allowed by contact at O₁ (Dashed
curved arrows)
470—Fields of Clockwise Rotational Freedom allowed by contact at O₂ (Solid curved arrows)
480—Fields of Counter-Clockwise Rotational Freedom allowed by contact at O₂ (Solid curved
arrows)
490—Fields of Clockwise Rotational constraint allowed by contact at O₂ (Dashed curved arrows)
500—Fields of Counter-Clockwise Rotational constraint allowed by contact at O₂ (Dashed
curved arrows)
510—Overall remaining field of Rotational Freedom (Clockwise within angle)
520—Tensioning cable
530—Normal force at O₁
540—Normal force at O₂
550—Cable tension pulling moving link with respect to fixed reference link
560—Pulse transmitter
570—Face of pulse transmitter
580—Grip of pulse transmitter
590—Coupling to control computer
600—Control computer
610—Treatment programming or selection
620—Treatment status display
630—Treatment delivery switch
640—Case of actuator
650—Mounting means for distractor to motor
660—Adjustment means
670—Anti-rotation means
680—Main drive shaft
690—Bearings
700—Lever arm
710—Ratchet mechanism
720—Return spring means
730—Nut
740—Threaded portions of shaft
750—Unthreaded portions of shaft
760—Key slot
770—Cable setscrews
780—Cable passages
790—Wrap spring clutches
800—Backstop wrap spring clutch
810—Return spring
820—Bushing
830—Pawls
840—Ratchet wheel
850—Base link
860—Stitch anchors
870—Spring arm
880—Spring arm
890—Wrap spring clutch
900—Drum portion
910—Bearing
920—Antirotation surface
930—Double ended shafts
940—Gearbox
950—Primary shaft
960—Secondary shaft
970—Staggered locations
980—Offset distractor parts
990—End Effectors
1000—Link Attachment member
1010—Bore hole
1020—Mounting Screw
1030—Fixation plate
1040—Locking setscrew
1050—Pedicle Screw
1060—Angular freedom compensation
1070—Axial compensation
1080—Rotational compensation
1090—Locking Collar
1100—Collar setscrew
1110—Slotted joint
1120—Side to side compensation
1130—Cover component
1140—Base component
1150—Mounting screw
1160—Mounting Screw hole
1170—Interlocking edge
1180—Tapped hole
1190—Face piece hole
1200—Flexible shaft
1210—Turnbuckle distractor
1220—Remote distractor
1230—Turnbuckle shaft
1240—Left and right hand threads
1250—Attachment points
1260—Prismatic joint chain
1270—Universal joint chain
1280—Threaded tension member
1290—Nut
1300—End loop
1310—Tension cable
1320—Rotary bearing
1330—Distractor links joint
1340—Distractor links
1350—Turnbuckle shaft
1360—Turnbuckle ends
1370—Turnbuckle nut
1380—Turnbuckle nut
1390—Articulated distractor
1400—Drive train
1410—Different threads
1420—Lever arm

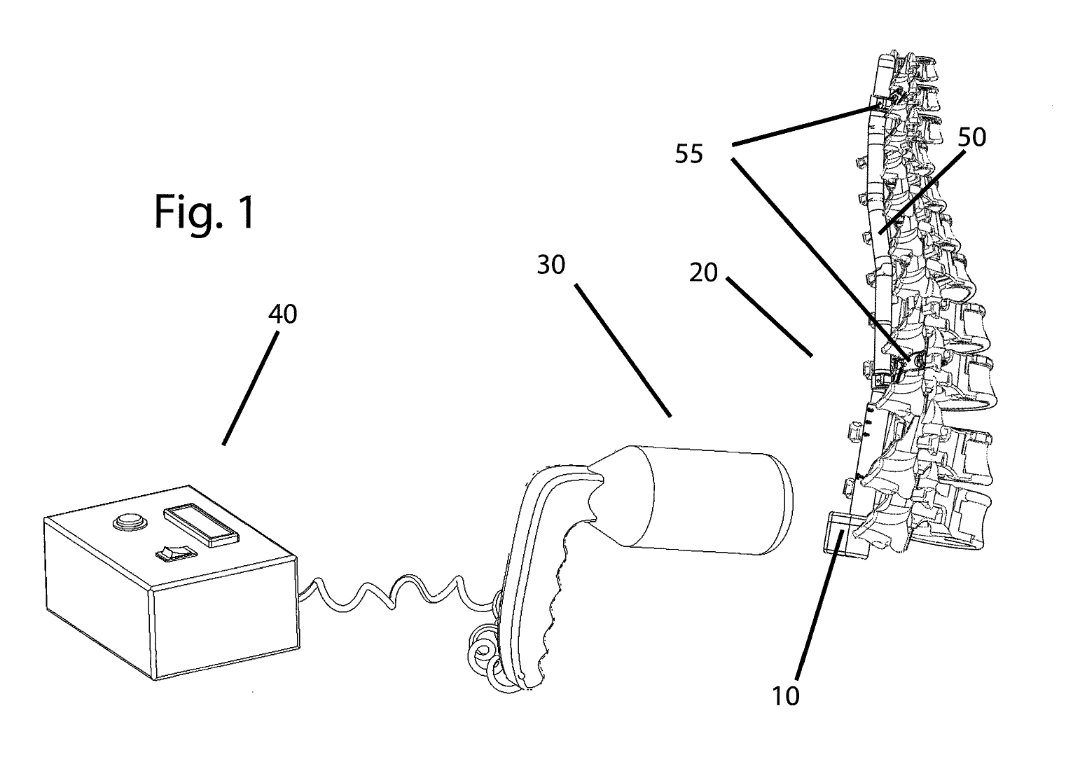

FIG. 1 gives a pictorial overview of key elements of the present system as it can be applied in correcting deformities of the spine.

Part 10 represents an implantable rotary or linear actuator that can be controlled by pulses delivered through the skin or other tissues of the patient.

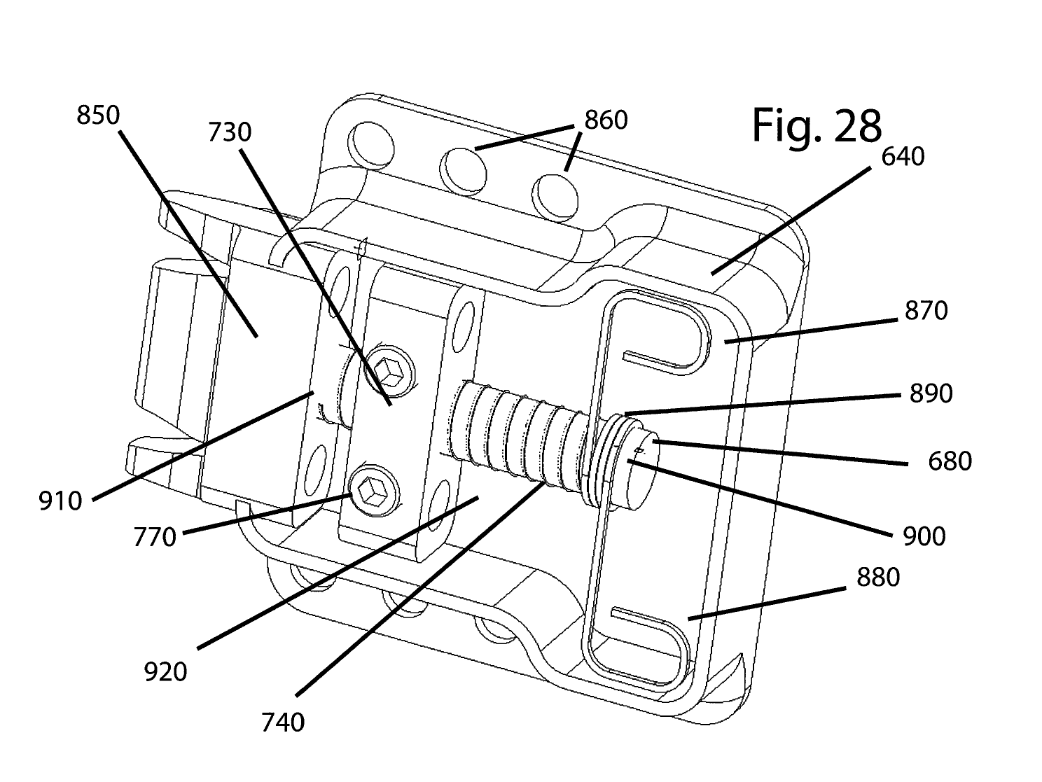

Typically, the number and duration of these pulses for a particular treatment can be planned and programmed into a control computer (part 40) and then delivered to the internal actuator 10 by a transmitting device such as an electromagnet, shown here as part 30. The pulses can be mechanically communicated to the internal actuator by pressure on the skin or through another intermediate implanted transmission medium such as a fluid filled bladder, such as by using the actuator of FIG. 28.

Part 30 is a pictorial representation of such a magnetic pulse source or other external transmitting device. It can be positioned in proximity to 10 but external to the patient. The alignment and air gap are such that a pulse from 30 can result in an incremental indexing rotation of the implanted actuator.

Part 40 illustrates the power, control, and computer system that can be used in conjunction with 30 to deliver a desired train of pulses to the actuator 10. Part 40 can incorporate computer or other means to allow the physician to preset such variables as the number of pulses to be delivered, their strength, their duration, and the timing between pulses. It can contain the trigger mechanisms for actually delivering the treatment pulses. It can also incorporate a display or other feedback mechanisms to convey to the operator the extent and status of the present treatment.

Part 40 also symbolically represents the power switching and control system that converts the low-level control logic information to the voltages and current levels needed to drive the treatment transmitter 30. Typically it can incorporate a solid-state relay, surge suppressor circuitry, and the like.

Finally, 20 typifies one of many possible deformable tissue distractor systems whose geometry can be gradually altered during the course of the treatment by means of the incremental actuator 10.

For instance, in FIG. 1, distractor 20 consists of a series of somewhat cylindric jointed link members 50 coupled together by internal cables. The joints between the somewhat cylindrical links are so disposed as to cause the link elements of distractor 20 to attain a predetermined final three-dimensional shape when the cables are tensioned. This shape correction is transmitted to the patient's tissues via a variety of end effector devices 55. FIG. 1 shows how the present system can be configured for treating scoliosis.

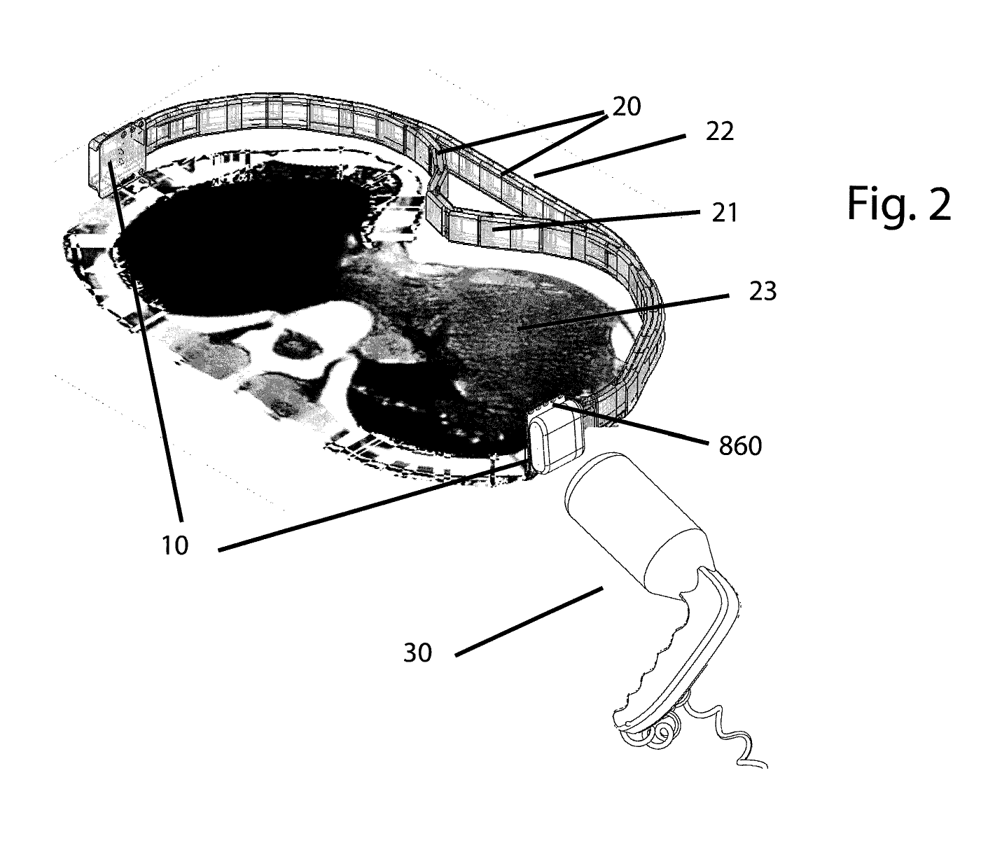

FIG. 2 shows how the present system can be utilized in treating pectus excavatum. (A transverse radiographic image of the patient's untreated sunken abdomen is shown in the background for context.) Here, the distractor 20 is comprised of a cable-tensioned system of jointed link members but they are flattened bar-like members rather than substantially cylindrical members as in FIG. 1.

In the case of pectus excavatum, this flexible cable-coupled system of links can be threaded beneath the sternum through small incisions proximal to the actuators 10 on either side of the patient. This can be facilitated using tapes or other guides. A flexible protective sleeve can be used over the cable distractor links to serve as a guide and also to prevent their accidentally pinching the patient's tissue. In some situations the cables can be introduced before the actual links to serve as guides. Then the individual links can be slipped along the cables, possibly working in from the two sides. The sleeve can have markings to aid a surgeon.

Mounting holes 860 in FIG. 2 represent typical means by which the distraction system can be sutured or otherwise anchored or attached in position.

In different applications of the present system, the length and curvature of the individual distractor links to be used can be preoperatively chosen by the physician so as to minimize trauma and obtain the best final geometry. Well-known imaging modalities and computer modeling can be developed to facilitate this preoperative treatment visualization process.

Shorter distractor links with joints closer together can be employed in the areas requiring the most correction of curvature. Similarly, distractor joints can be spaced further apart in areas where less correction is needed.

After insertion, the cables and links can then be pre-tensioned and fastened to anchor nut devices on the incremental surgical actuators 10. FIG. 1 shows an application with a single such actuator device for correcting spine curvature. FIG. 2 shows a chest wall deformity application using a pair of these actuators 10 attached beneath incisions on either side of the torso.

Postoperatively, the incremental actuators can be progressively tightened over a period of time. The present system teaches how the geometry of the mating surfaces between the links can cause them to gradually move into a preferred final alignment with one another. As the joints approach their final alignment, the distractor system becomes stiffer so it can better resist bending. As the transition takes place, the end effectors attached to the links of the distractor gradually apply increased corrective forces on the anatomical structures. This gradual change can alleviate much of the pain and discomfort associated with present more abrupt surgical treatments.

FIG. 2, for example, shows how the present system can gradually make such a transition. At the start of treatment, the un-tensioned distractor can follow the general shape of the patient's chest wall as shown in initial configuration 21.

During treatments spread out over a period of time, when the physician activates the pulse generator 30, the incremental rotations of the actuator 10 increase the tension in the cables and cause the distractor device 20 to transition from a starting configuration 21 compatible with and somewhat conforming to the initial shape of the deformity being treated to a final desired shape such as 22.

In the illustrative example of pectus excavatum treatment (shown in FIG. 2) the sternum and ribs can be gently forced into a more natural orientation near the end of treatment. Once the tissues had adapted to the new chest wall configuration, the link distraction system can be surgically removed.

The present system differs from other devices because this change in configuration is primarily due to angular bending in the joints between adjacent cable connected link elements and not due to telescoping of said links.

As the distraction device makes this shape transition, the distractor 20 will provide forces on the actual tissues of the patient via a variety of end effector elements 55 attached to said distractor links. These end effectors are chosen and arranged so as to preferentially coerce the tissues in desired directions thereby providing correction to the deformity.

These end effectors 55 serve to interface the progressively reshaping distraction apparatus to the patient's tissues. End effectors 55 can comprise such well-known devices as surgical screws or other pedicle attachments. Specialized end effectors 55 are also described in and as part of the present system.

End effector action in the present system can also take place as a result of shaped surfaces or coverings on the distractor link elements themselves that can bear directly against the tissues. This mechanism for tissue distraction is employed as shown in FIG. 2.

In FIG. 2 for example, the outward facing flattened faces of the jointed links of distractor 20 serve as the end effectors. These bearing surfaces are so disposed as to push outward on the chest wall when the cables are tightened, thereby correcting the deformity when treating pectus excavatum.

The final shape that the stiffened distractor 20 will attain is controlled by the chosen distractor link shape and dimensions, the tension cable arrangement, and by the joint geometry. Custom or standardized distractor 20 and end effector components can be chosen or manufactured to meet the particular needs at hand and forces required.

The present system teaches how a variety of joint geometries can be selected so as to produce desired forces, rotations and translations at various points along the distractor 20. Some joints can have stops allowing limited angular rotations, say, while others can permit free motions in certain directions but constrain motion in other directions. In this manner the final post-tensioned shape and curvature of distractor 20 can be controlled along its length in three dimensions.

FIG. 3 shows an exemplary embodiment of the present system using a pair of tensioned cables 60 to draw the links 50 into alignment. The cables 60 are attached to a common draw nut assembly at the actuator end and a common actuator or anchor means at the other end so they act in concert to draw the links 50 into tighter contact with one another and with a desired final alignment.

As the slack is taken out of the system, adjacent pairs of links will be drawn together as at 70. The cables running through lengthwise passageways 74 in the links also press against and are in contact with various areas on the periphery of the passageways through the links.

Due to the small clearances, the net effect of this fairly complex interaction of slipping, sliding, and rubbing between parts will be to coerce the links towards a configuration where the contacting surfaces 72 between adjacent links are pulled towards coincidence. Effectively there will be an approximate hinging motion between the links with the pair of cables acting to constrain the direction of this hinge axis as shown by arrow 76 in FIG. 3.

FIG. 3 also shows how the links of the present system can be either straight or curved. They can also be twisted in three dimensions, so as to produce distractions in multiple planes.

FIG. 3 further shows how the links can have flattened bar-like cross sections if that is beneficial, say for strength reasons or to better fit the patient or to provide more comfortable bearing surfaces against anatomical structures. Link edges can be rounded with fillets as shown and the links and joints can also have a flexible protective tubular sleeve or cover to prevent tissue from growing or being pinched in the joints.

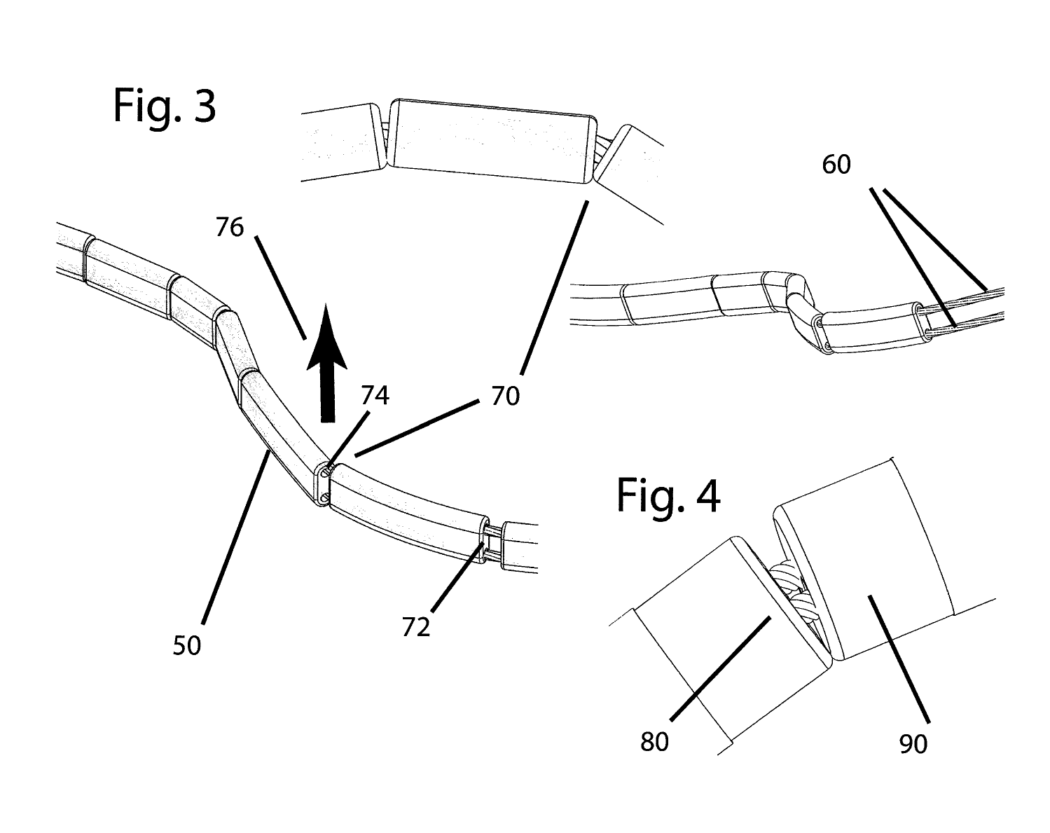

FIG. 4 shows how the links of the present system can also be constructed with a cylindrical or other cross-section.

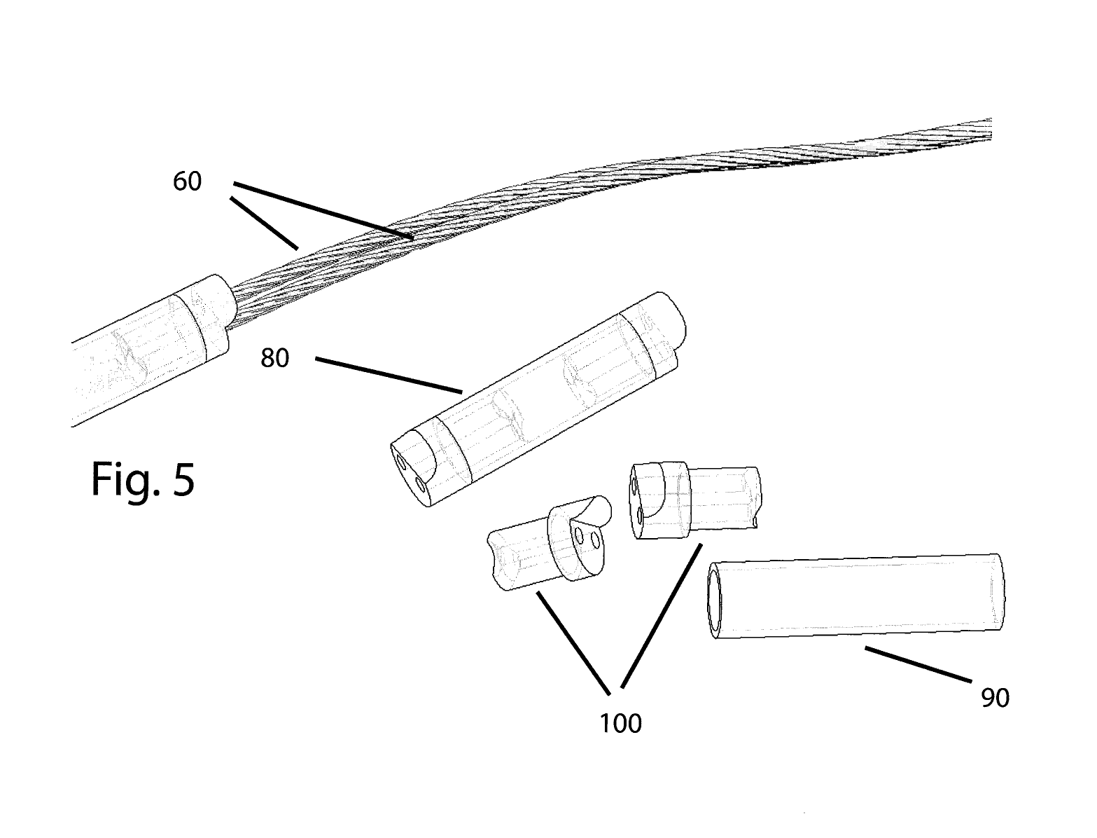

FIG. 5 shows a joint for the present system that will aid the links in following a desired hinge axis as they come into alignment. Here, the two halves of joint 100 have mating male and female sliding surfaces that align the link bodies and guide the hinging action as the cables are tightened.

FIG. 5 also shows how the joints of the present system can either be made as integral parts of the links or as separate components attached to and functioning as part of the links. 80 gives an example of a complete link assembly with a tubular link body 90 and joint halves such as the male and female pair 100 attached at both ends. Such a construction can allow a smaller inventory of components to be customized to meet the needs of a particular patient. For instance, the tubular bodies 90 can be stocked in various lengths or bends and combined with standard joints 100. The joints at the two ends of 80 can be pressed or keyed into the tubular members 90. They can also be rotated slightly with respect to one another to provide out of plane distraction.

The joints can also have their hinge axes oriented in different planes and have their colliding surfaces so arranged as to provide a desired amount of spatial rotation at each joint. They need not be manufactured to result in an in-line orientation of the coupled links but can be customized as needed for a particular distraction purpose. Further, modifications can be incorporated within the spirit of this system, for instance to allow one or more cables or multiple passages for cables.

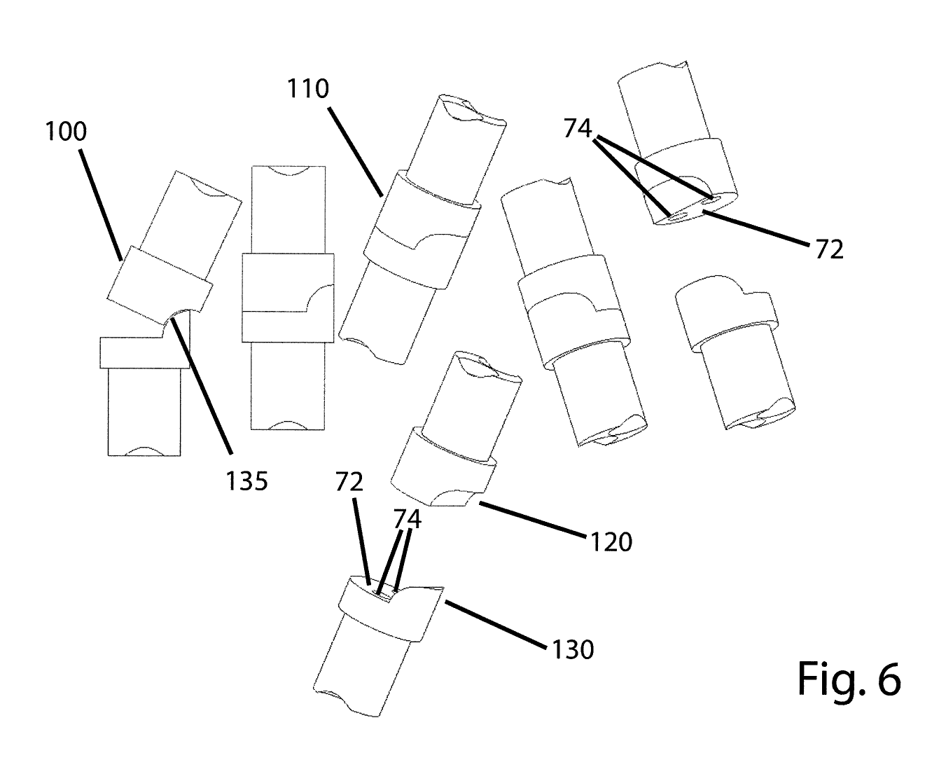

FIG. 6 details how the joints of the present system can be made with male and female portions 120 and 130. These portions can have sliding surfaces 135 in the form of partial cylinders that will guide the attached links with a hinging action. When the cables are fully tensioned and the links are in their final positions, this hinging action is arrested by the substantially flat surfaces 72 coming into face-to-face contact. The part-to-part contact between the sides of the cables 60 and the axial passageways 74 through the joints will result in a strong, relatively stiff final configuration where side-to-side loads on the distractor of the present system will be primarily absorbed as shear loads in the cables rather than as tensile loads. For clarity, the cables are not shown in the FIG. 6 illustration but are understood to run through the passageways 74.

It will be appreciated that a variety of mating surface geometries can be used for the two halves of the joints to control the final tensioned positions of the cable-coupled links. Further, a multiplicity of cables can be used as shown in FIG. 6 or a single cable can be employed to pull the halves of the joints into alignment.

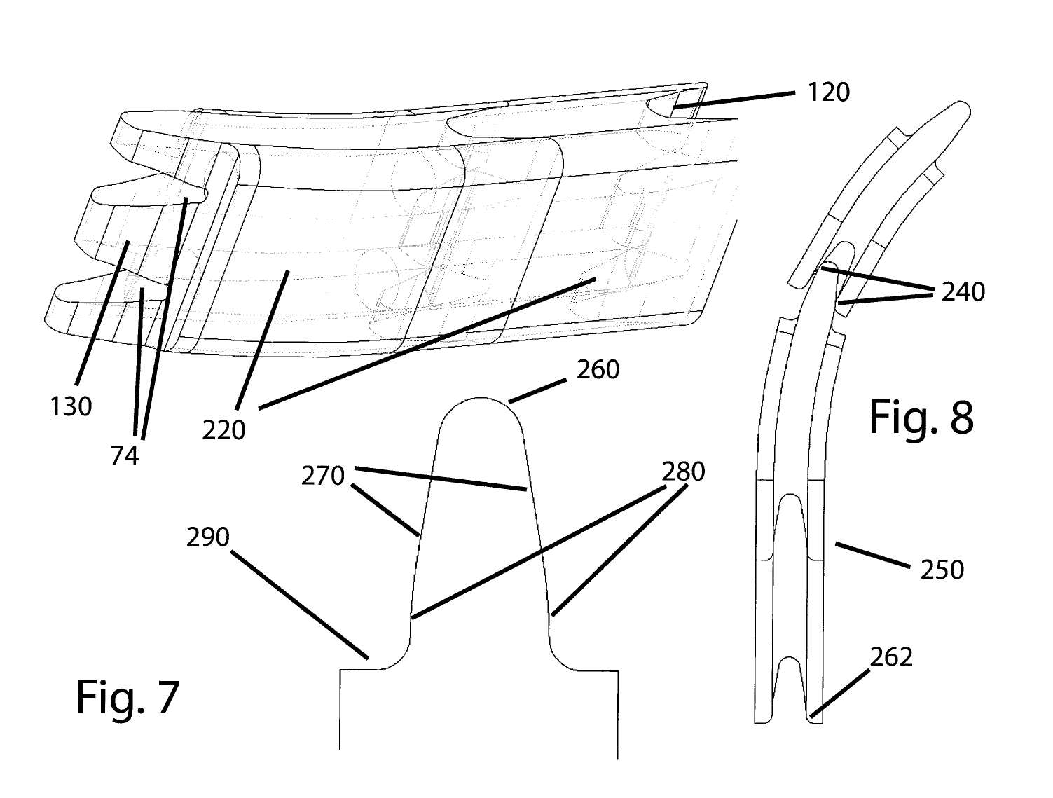

FIGS. 7 and 8 show other variations for constructing the joints of the present system. The joint geometry shown in FIGS. 7 and 8 provides positive spatial alignment between adjacent links, correction of misalignment, and higher bending strength then can be otherwise achieved by depending on the cable tension alone. This joint geometry can be particularly well suited to distractors for the treatment of pectus excavatum or pectus carinatum, as illustrated in FIG. 2.

The links shown in FIGS. 7 and 8 have male and female mating coupling surfaces. As seen in the cross-sectional projected views, the coupling surfaces of are characterized by rounded surfaces 260 and 262, and wedge-like surfaces 270 which make a smoothly curved transition into parallel flat surfaces 280. (It will be appreciated that the cables that draw the links together are omitted from FIGS. 7 and 8 for clarity. These cables pass through the cable passages 230.)

When loosely assembled so as to follow the curvature of the deformity at the time of surgery, the coupling surfaces of one or more adjacent links can be only partially engaged as seen at 240. Line or surface contact can occur, say between the male or female rounded surfaces 260 or 262 and the male or female round or flat surfaces as seen at 240.

As the cables are tightened, either because they were shortened by means of the ratchet actuator or because of motion or growth of the patient, vibrations, or other disturbances, the geometry shown in FIG. 7 forces adjacent links into proper alignment. Wedging action between the joints straightens the connections and amplifies the effectiveness of the cable tension in resisting side bending loads on the joints. Finally, once the mating parallel surfaces 280 are engaged, the cable is relieved from providing any of the bending stiffness to that portion of the distractor link assembly. The cable's main function then is to simply keep the links engaged.

FIG. 7 shows a preferred configuration with provision for two cables running through the passageways 230 shown. One or more cables can be used within the spirit of this system. Again, for clarity of illustration, the cables have been omitted from the FIG. 7 illustration.

It will be seen that the cable-connected links 220 in FIG. 7 can be made in different lengths and different curvatures to meet the needs of a particular patient or operation.

Further, one can employ a variety of attachments and fittings on the various links 220 as needed for such purposes as providing intermediate stiffening, support, stabilization, suture attachments, and so forth.

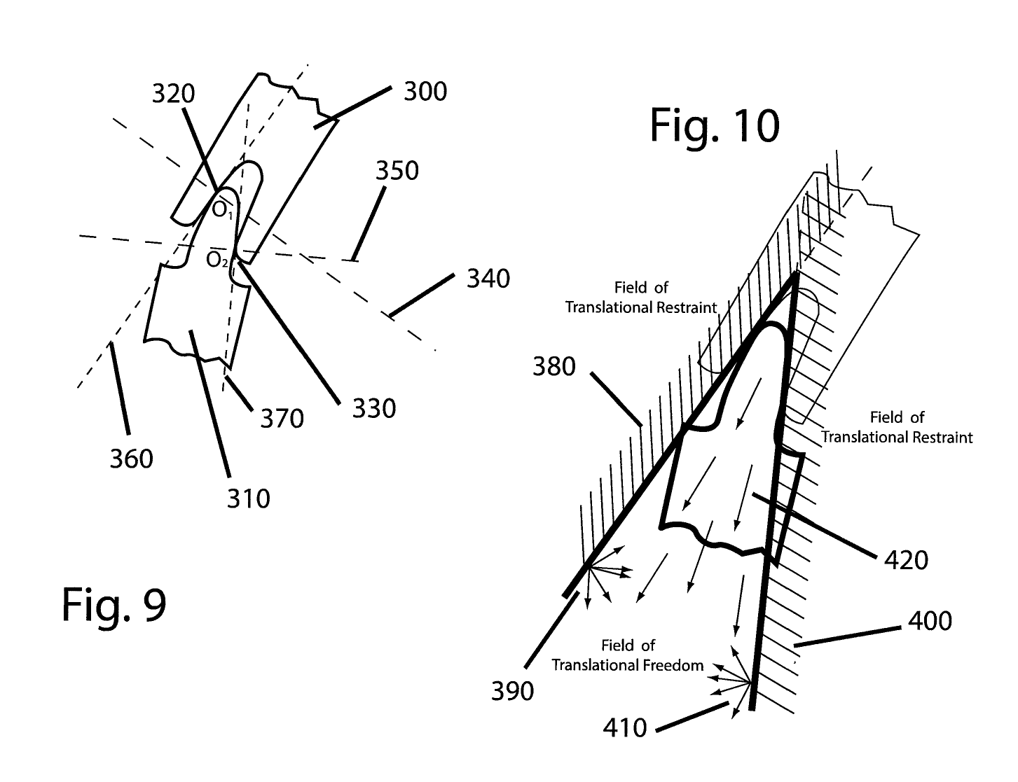

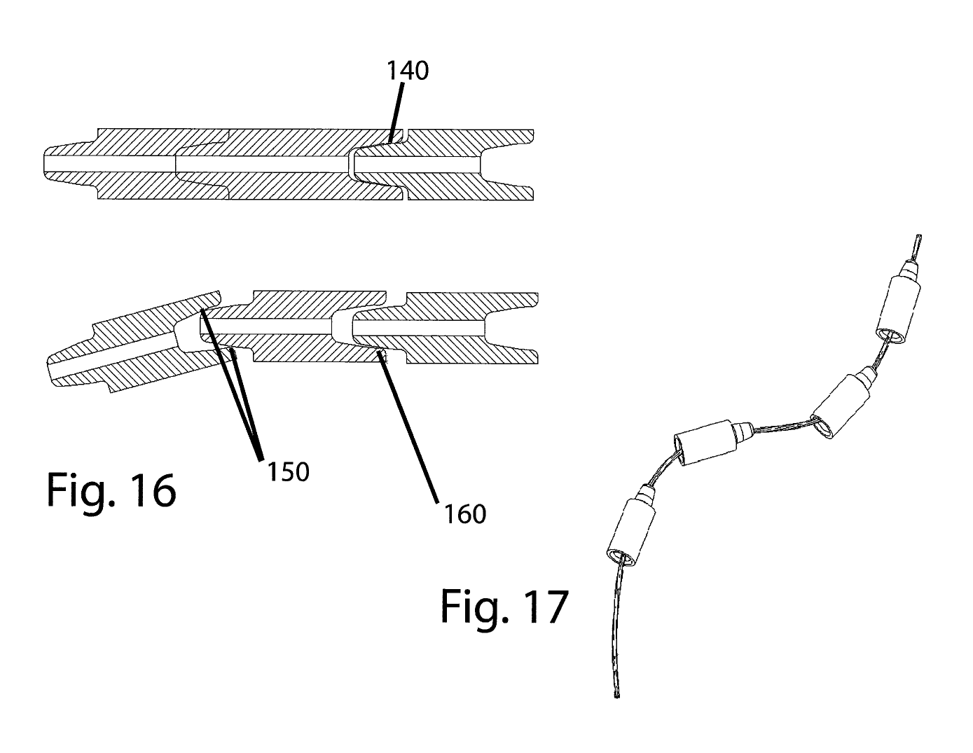

FIGS. 9 through 15 teach in two-dimensions several underlying theoretical principles of the present system. These joint design principles can be extended into three-dimensional geometries for other distractor applications as is shown in FIGS. 16 through 18. In FIGS. 9-15 the common normals and common tangents at the contact patches between links control the relative positions of the links and how they can move as the cables are tightened in the present system.

It will be understood that the two links 300 and 310 of FIG. 9 are being pulled together by tensioned cables running through passageways within the links. These passageways run substantially down the centerline of the links as seen in these views and in the FIG. 8 projected view. The coupling cables and passageways are not shown in these diagrams for reasons of clarity.

When the cable is snug and the joint is partially assembled as shown in FIG. 9 there will typically be contact between the male and female portions of the joint at several points such as 320 and 330. The number of these contact points together with their locations and inclinations relative to one another determines the relative motions possible between the two links 300 and 310.

As seen in FIG. 9, at each contact point one can construct an instantaneous common normal to the two halves of the joint and a common tangent. At 320 the common normal is 340 and the common tangent is 360. At 330 the common normal is 350 and the common tangent is 370.

Considering just the instantaneous contact between the two bodies at point 320 of FIG. 9, it will be seen that the only directions in which body 310 can move relative to body 300 is shown by the illustrative arrows 390. Motion towards the other side of the common tangent at 360 is blocked by the fact that the two links would collide at 320 if link 310 were to try to move in that direction. Given this one point of contact at point 320, motion in any of the directions suggested by the arrows 390 is possible because it would tend to open the gap and separate the contact at 320. Thus on one side of the common tangent at 320 we can say there is a “field of translational freedom” and on the other side is a “field of translational restraint”. This instantaneous “field of translational restraint” caused by the contact at 320 is shown by the cross-hatched region 380.

If we have two or more simultaneous points of contact (320 and 330 in this case) then each common tangent produces a field of possible translational freedom (fields 390 and 410) and a field of translational restraint (380 and 400). Whether or not one body can translate relative to the other is determined by whether or not there is any residual field of translational freedom once all such contact points have been taken into account.

FIG. 10 shows how the two contact points shown in FIG. 9 limit the ability of link 310 to translate relative to link 300. At this instant, link 310 can only translate out of the wedge-shaped region shown. The cross-hatching shows the fields of translational restraint in which one or the other or both contact points are colliding thereby preventing translational motion.

If the links were initially separated but being pulled together by a cable then the rounded nose of link 310 can translate towards the female socket region of link 300 until contact occurred between the two links, say at point O₁ or O₂. When that happens, sliding along the common tangent at the contact point can further guide the moving link within the field of translational freedom towards the nested configuration until a second contact point became active and further translation was blocked as is shown in FIG. 10. Tension in the cable can prevent translation out of that residual wedge-like field of translational freedom so all translatory motion would be blocked once the two contacts at O₁ and O₂ occurred.

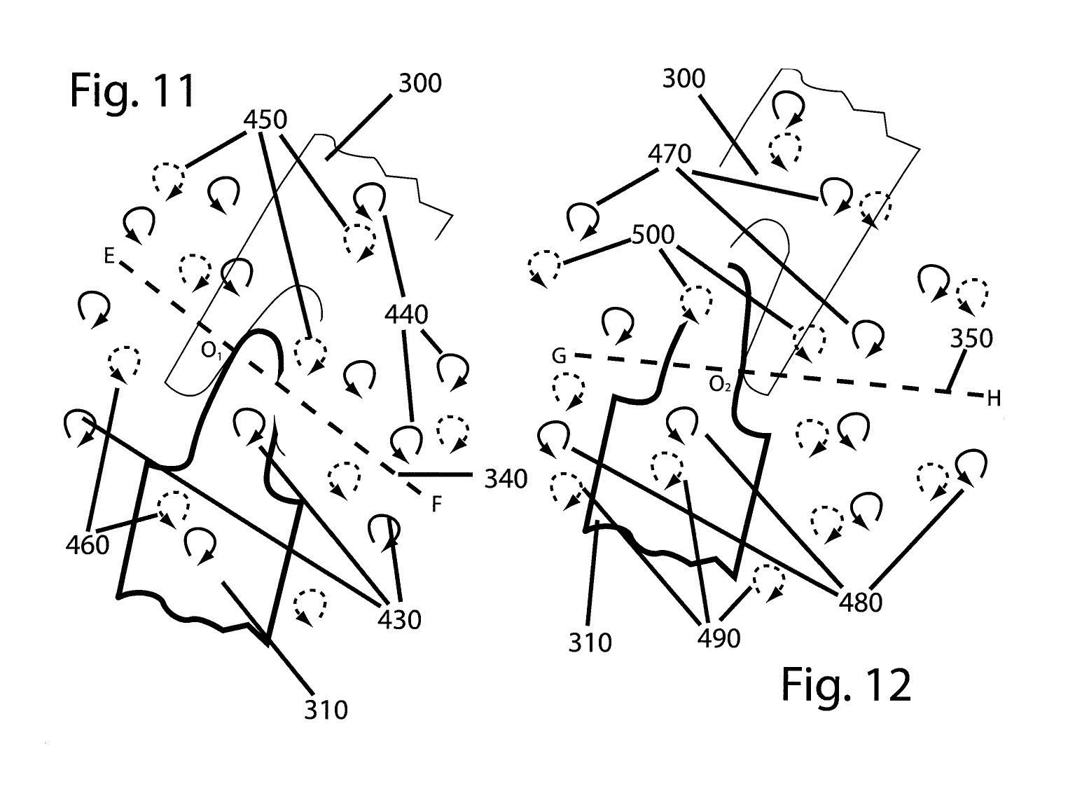

Similarly, FIGS. 11 and 12 teach how the common normal between two colliding bodies determines how they can rotate relative to one another. For instance, in FIG. 11 again consider the upper body (300 shown here with the light outline) as being the fixed reference body and see how the contact at point O₁ limits the ability for the lower body (310 shown here with the heavy outline) to rotate relative to the reference body. (Ignore for the moment the contact at point O₂.)

The common normal at the contact point O₁ is the line 340 (E O₁ F). Clockwise or counter-clockwise rotation of body 310 relative to body 300 is possible or restricted depending on whether the instantaneous center of rotation is located on one side or the other relative to this common normal.

In FIG. 11, if the center of rotation is above the normal 340 then a counterclockwise rotation of the body 310 is possible because rotation in that direction would tend to open the gap at O₁. In other words, there is a counterclockwise field of rotational freedom as shown by the solid curved arrows sketched above line 340.

Similarly, if the center of rotation is below the normal 340 then a then a clockwise rotation of the body 310 is possible because it would also tend to open the gap at O₁. FIG. 11 shows this field of clockwise rotational freedom by means of the solid curved clockwise arrows in the region below the normal 340.

Conversely, body 310 is blocked from rotating in the clockwise direction about any center of rotation lying above the normal 340 because such a rotation would tend to bring the contacting surfaces closer together into a collision at O₁. The dashed clockwise curved arrows of

FIG. 11 show that clockwise rotation is impossible about centers of rotation in that region. We can refer to the region above the common normal 340 as the “field of clockwise rotational restraint” produced by contact at O₁.

Similarly the region below the common normal 340 is the “field of counterclockwise rotational restraint” produced by contact at O₁, as shown by the counterclockwise dashed curved arrows of FIG. 11.

FIG. 12 shows the similar rotational constraints produced by the contact at point O₂. Common normal 350 divides the plane into a region of clockwise rotational freedom above line 350 and counterclockwise rotational freedom on the other side of the common normal. Similarly, contact at O₂ causes a field of clockwise rotational restraint below the normal 350 and counterclockwise rotational restraint above the normal.

All of these fields are instantaneous properties and change as the contact points shift position and direction.

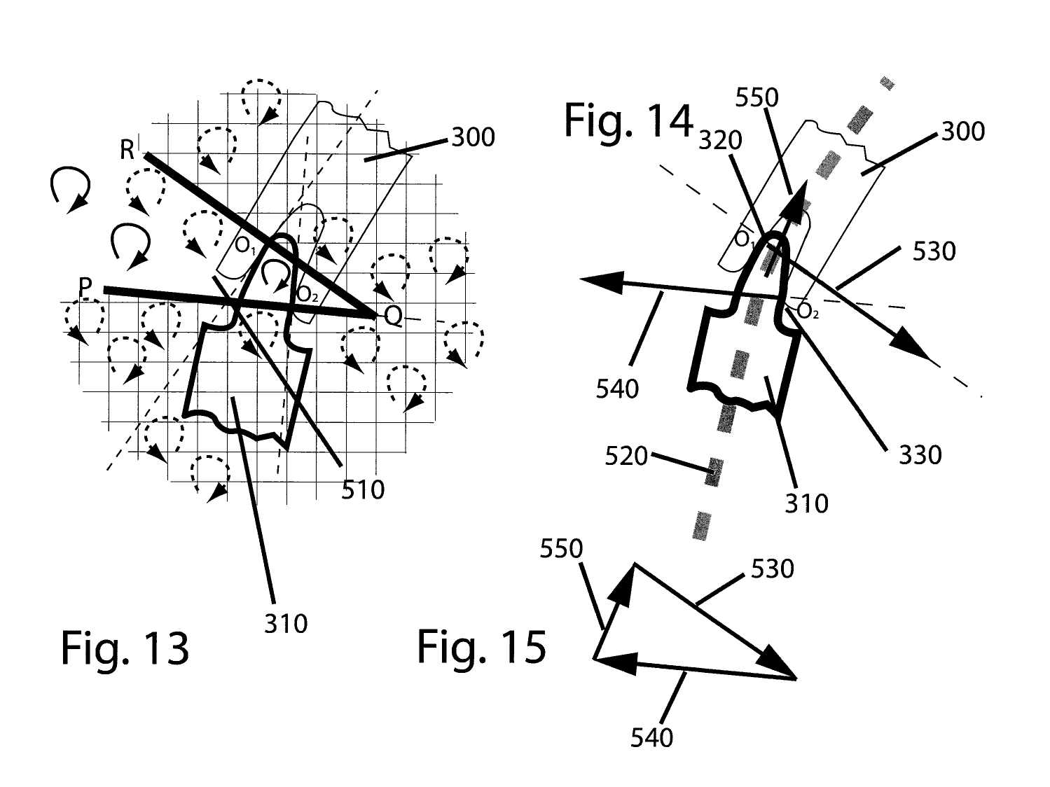

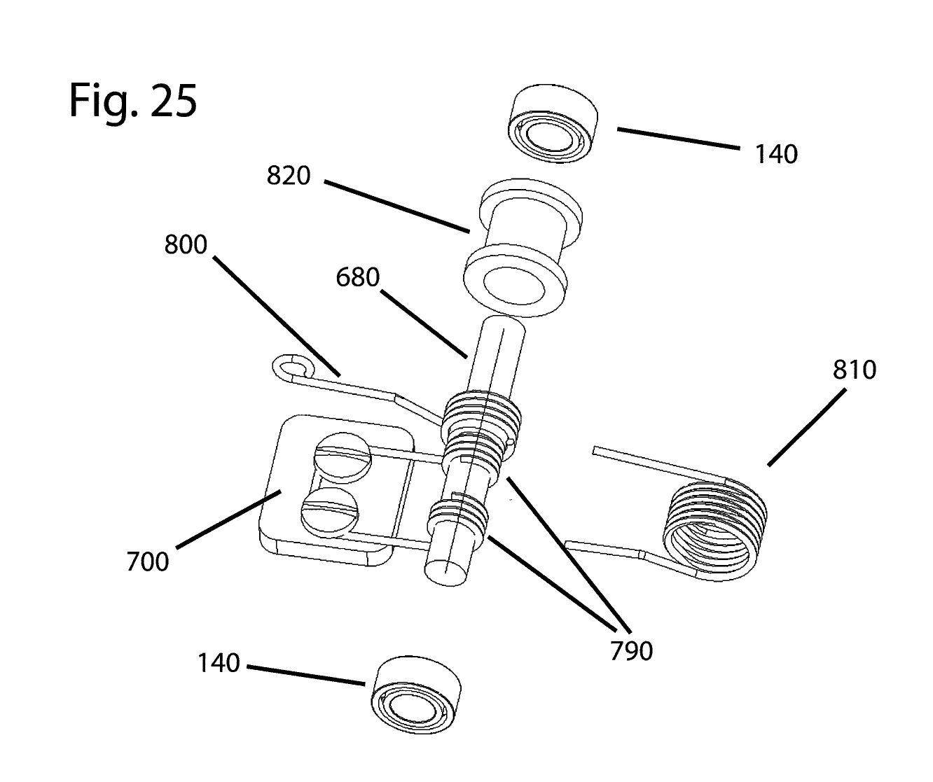

FIG. 13 shows that with simultaneous contact between links 300 and 310 at the two points O₁ and O₂ shown in FIG. 25 these common normals and common tangents define overlapping regions of clockwise and counterclockwise rotational freedom and restraint. In order for body 310 to rotate at all it must rotate about a center of rotation in a field of rotational freedom that is not blocked in both the clockwise and counterclockwise directions.

FIG. 13 shows that body 310 can only rotate in a clockwise direction relative to body 300 and only about a center of rotation inside the acute angle P Q R defined by the instantaneous common normals at the two contact points. All other rotations are blocked.

When contact at O₁ or O₂ temporarily prevents the male link 310 from translating further into a nesting position with the female socket in link 300 it forces link 310 to rotate into alignment in response to tension from the cable. Similarly, when link 310 has rotated into proper alignment and there is no further field of rotational freedom, the parallel tangent surfaces defining the field of translational freedom will allow it to be pulled further into the nesting position until it is firmly seated and additional contacts block further nesting motion.

Thus, the geometry illustrated by this provides that the joint can only move in such a way as to close the joint and rotate into proper alignment when the joint halves are constrained from separation by a tensile cable. Since there is no side-to-side field of translational freedom once the links are nested, the effective side-to-side bending strength of the joint is primarily a function of the strength of the link materials and thicknesses and not of the tension in the cable.

This closing of the gap between the two halves of the joint can be urged or caused by the tension in the cable but it can also result from any other disturbance such as random jiggling motions within the joint. However, once it occurs, if the cables are able to hold the joint from again separating then the only possible motions will be in the desired direction and tend to close the joint.

FIG. 14 is a simplified free-body diagram showing the principal forces active in the joint just discussed during the period before the links are fully engaged. Vector 550 represents the cable tension pulling link 310 towards link 300. Vector 530 represents the normal force which link 300 is exerting on link 310 at contact O₁. Vector 540 represents the normal force link 300 is producing at contact O₂.

It will be seen that forces 530 and 540 produce a clockwise couple tending to rotate link 310 into alignment with the joint.

FIG. 15 is a polygon of forces showing the force equilibrium between these forces. (Frictional forces have been ignored for clarity.) It shows that the construction taught by the present system can produce or resist fairly significant side forces 530 and 540 with a relatively small cable tension 550. As the links come closer to being in alignment the tensile loads in the cable (and that must be produced by the actuator) dramatically decrease.

These same general principles apply to joints constructed by the teachings of this patent when extended to three dimensions, say by rotating the two-dimensional profiles of FIG. 9 about an axis so as to form a swept male or female joint somewhat like a wine glass or goblet, as illustrated in FIGS. 16-18.

FIG. 16 illustrates how these principles taught in connection with the FIG. 9 planar joint also work when generalized and extended into three dimensions. The analogy with the planar device is as follows: In three dimensions, the points of contact between the joints become contact patches and the tangent lines become tangent surfaces. The spatial translational and rotational freedoms permitted by the joints are now constrained by half-spaces defined by the common tangent planes and by the directions of the common normals at the contact points.

When the planar joint geometry of FIG. 9 is swept about an axis into a surface of revolution, links such as those shown in FIGS. 16 and 17 can be generated. Those links will be constrained by the contact patches between the male and female joint components to rotate and align into a stiffened member as the cables are tightened. However they can still have a rotational freedom about an axis aligned with a cable passing through the joint. Depending on the specific application of the present system that extra freedom can or can not be important.

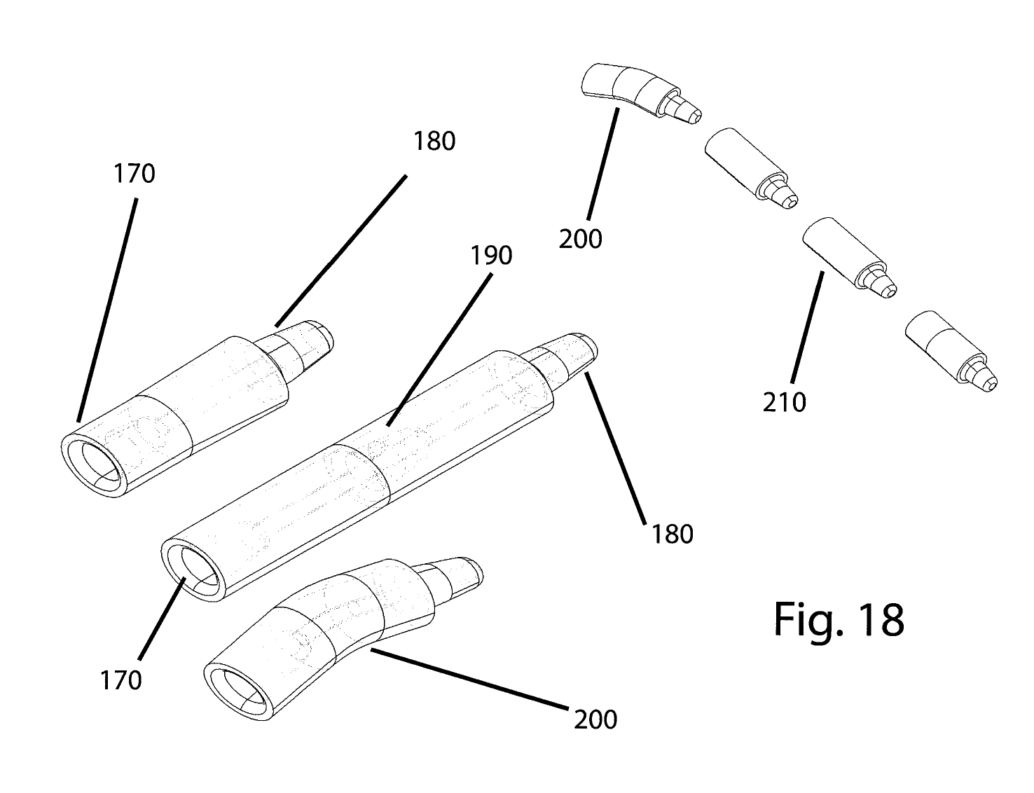

FIG. 18 shows how that remaining rotational freedom can be removed by sweeping the planar joint about a non-circular cross-section such as an ellipse. Here, the male joint half 180 and the female joint half 170 are shown as having elliptical cross-sections although other mating non-symmetrical geometries can also work. Such joints can nest or key together in a manner that can eliminate all relative freedoms of motion as the cables pull the joint halves together. Other asymmetric geometries can also be used within the spirit and teaching of this patent to cause the joint haves to key into alignment as they pull together.

The remaining rotational freedom can also be removed by employing two or more tensioning cables running through side-by-side passageways as shown in FIG. 5.

In addition, as the joint closes into the regime in which the plug and socket are substantially prismatic rather than conical, the bending stiffness of that portion of the distractor is greatly increased. There is an increasing wedging action that enhances the mechanical advantage of the distractor as was taught in FIG. 15. The system makes a transition from producing distractor forces primarily because of the tension in the cable and the effective lever arm at which the cable tension acts to a configuration where the distractor can produce or resist forces primarily because of the geometry and material properties of the links themselves. When the joint is fully drawn together, the cable tension is only needed to resist axial loads and maintain axial assembly of the distractor links.

FIG. 18 also illustrates how the links of the cable-tensioned distractor can be bent (as shown by link 200) or twisted along their lengths as needed to achieve distraction in different planes and directions at different joint locations.

FIGS. 19 through 22 show suitable electronic and control circuitry for driving the imbedded actuator 10 of the present system. Other circuitry can also be used.

FIG. 19 shows a representation of how a physician can actuate the present system during a distraction treatment. The physician can initiate an incremental reshaping of the distractor 20 by placing an external transmission device 30 in a position where it can communicate one or more pulse signals to the implanted incremental actuator 10. The physician can use a control system represented by 40 to send a desired series of pulses to the implanted actuator 10. Suitable electronic computer and control circuitry can be integrated with and activated by parts 30 and 40.

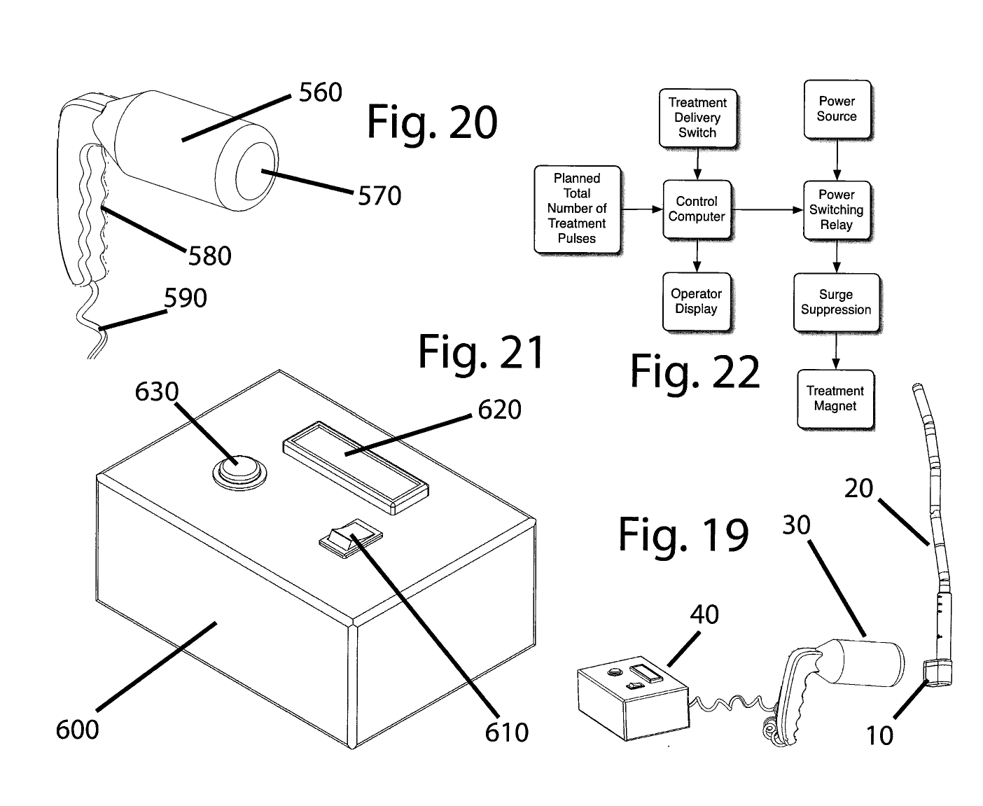

FIG. 20 represents a typical external pulse transmitter 30 that can be used with the present system. It will be appreciated that the particular shape shown in FIG. 20 is unimportant but is just used for illustrative purposes.

The shape of transmitter 30 is such that it can be positioned by means of a handle 580 or other device with a face 570 near or against the skin of the patient in the vicinity of the implanted rotary ratchet actuator 10. Control switches and other devices can be incorporated into the unit as desired along with needed safety devices such as thermal and electrical insulation.

Transmitter 30 can have a first surface 570 so disposed as to aid in properly positioning the pulse generator 560 with respect to the implanted actuator.

Transmitter 30 can comprise an electromagnet with windings and pole pieces capable of producing a magnetic pull strong enough to act through the tissues and air gap near face 570 and thereby index the implanted magnetically activated ratcheting rotary actuators 10 taught in this patent.

It will be understood that other forms of transmitter device can be used within the spirit of this system to drive other forms of implanted actuator motors. These can include wireless transmitters to drive implanted actuator motors with internal power supplies, induction coils to transmit pulses to implanted receiving coils, and other devices.

Transmitter 30 can also have a handgrip or other means 580 suited to aiding the physician in aligning and positioning the device with respect to the patient and a means 590 for connecting device 30 to necessary power and control devices 40. It will be understood that elements of 40 can be integrated into 30. They are shown as separate component modules simply for illustrative purposes.

Switch 630 of FIG. 21 represents a typical means that can be used by the physician to deliver a predetermined number of electromagnetic pulses to the implanted actuator. An associated microprocessor or computer (represented by box 600) can be programmed to convert the desired treatment protocol from terms most useful to the physician to the needed number of pulses to drive the implanted actuator. For instance, the physician can specify to the computer the length of cable to be retracted by the actuator and the computer can convert that into the number of pulses needed to achieve that distraction.

As an example, a display means 620 can be used as a human interface to aid in presetting the number of pulses to be delivered, to indicate the number of pulses currently delivered, or to graphically show the status of the present treatment. Such a display means can optionally even be coupled to real-time imaging hardware to show how the patient or the implanted distractor system is actually responding to the current treatment.

Similarly, there can be interface devices coupled to the control computer to change the planned treatment. For instance, devices like switch 610 can be used to preset such things as the number of pulses to be delivered, their timing or duration, the total angular rotation to be attained, or the linear distance through which the distractor is to move. Sensors and feedback devices can also be incorporated within the distractor system to communicate to the external control system 40 internal parameters within the patient such as tension in the cables or forces on the distractor.

FIG. 22 is a flow chart schematic showing the logic and general arrangement of how the control system 40 and electromagnet 30 of the present system can be configured.

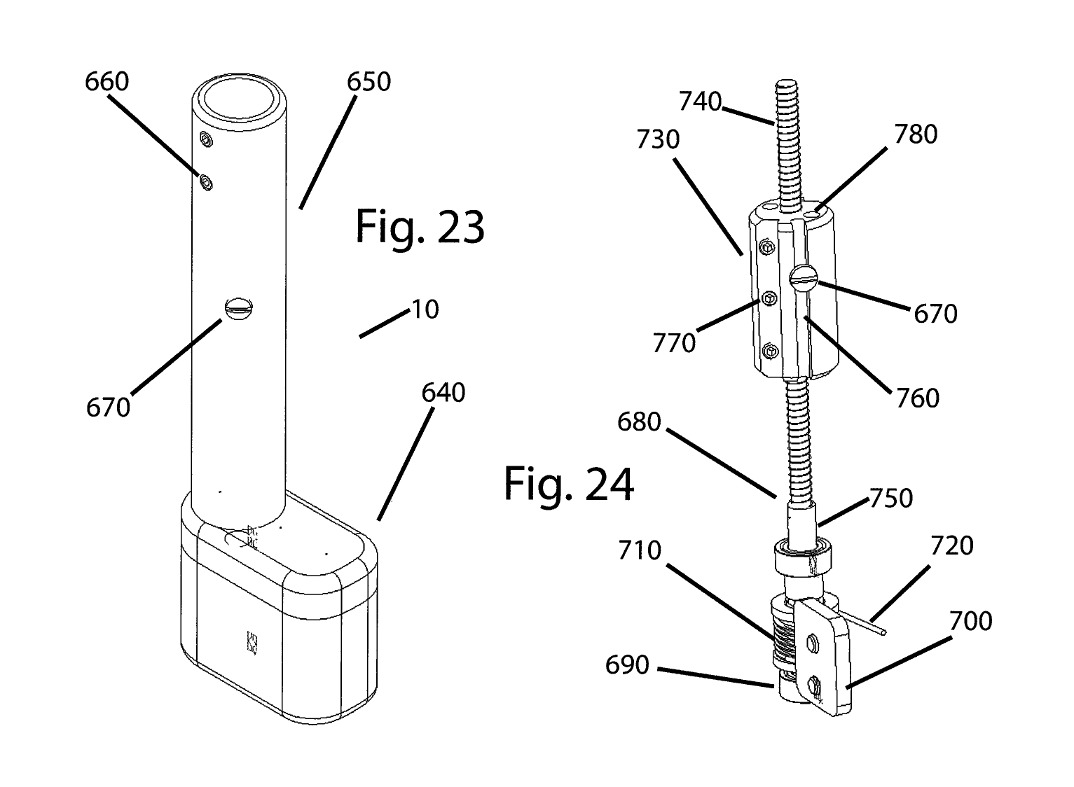

FIG. 23 shows elements of a typical actuator of the present system. Here, 640 is the outer non-magnetic housing or case of a rotary incremental actuator 10 operated by the external pulse source 30. Part 650 shows a possible mounting means via which said actuator 10 can be attached to distractor 20.

Setscrews 660 in FIG. 23 represent a possible assembly and adjustment means by which the relative positions of actuator 10 and distractor 20 can be initially adjusted as needed during the open surgical procedure to implant the present system.

Part 670 represents a typical anti-rotation means by which the rotation produced by actuator 10 can be blocked from transmission to other parts of the system. It shows one means by which reaction forces can be absorbed internally within the system.

FIG. 24 shows an internal view of actuator 10. 680 is a shaft rotationally mounted on the actuator case via bearings 690. Shaft 680 has threaded portions 740 and unthreaded portions 750 of various diameters. Said bearings 690 allow shaft 680 to rotate but provide thrust resistance along the axis of the shaft. 700 is a lever arm made of ferrous or other material that can be magnetically pulled by external electromagnet 30. 710 shows a ratchet mechanism whereby angular rotation of arm 700 can be transmitted to shaft 680. 720 is a return spring means capable of returning arm 700 to its rest position when the magnetic field is removed.

In FIG. 24, 730 is a nut assembly threaded onto shaft 680 and so disposed as to convert the rotation of shaft 680 into axial motion of the nut assembly 730 along the shaft. Suitable keying means such as pin 670 sliding in a groove 760 in the nut are used to prevent the nut from rotating with the shaft. Setscrews or other means 770 are used to couple the translating nut to cables or other devices moving elements of the distractor 20 relative to the actuator body 640. In FIG. 24 the cables can be held in the nut by means of the cable passages 780 through which the ends of the cable can enter the nut assembly and by the setscrews 770 pinning them within the nut assembly. Other well-known cable clamping means can also be employed to hold the cables to the nut assembly.

FIG. 25 is an exploded view of the inside of actuator 10 which uses wrap-spring clutches as an illustrative example of the ratchet actuator principles taught in the present system.

Arm 700 pivots around shaft 680. Wrap spring clutches 790 produce incremental rotation of shaft 680 when arm 700 swings in one direction but allow the arm 700 to rotate independently of shaft 680 during the return swing. Backstop wrap spring ratchet 800 acts between the case 640 and shaft 680 and is used to prevent shaft 680 from being rotated in the reverse direction. Torsional return spring 810 is used to reposition the arm between pulses. It will be appreciated that other return means can be employed. Here, bushing 820 provides bearing surfaces to prevent the various mechanism components from jamming.

It will be appreciated that other ratchet means can be employed in place of the wrap spring clutches illustrated in FIG. 25. The wrap spring clutches are particularly advantageous, however, due to their compact size and their ability to function with small input swing angles of arm 700. Further, rectangular shaped wire can be advantageously used for the wrap spring clutches, to minimize the tendency of the clutches to try to move axially on the shaft 680.

Other well-known ratchet means can be substituted for the wrap spring drive clutches 790 or for the wrap spring back stop 800. Some such devices include: toothed ratchets and pawls, sprag clutches, roller clutches, multi pawl ratchets, spiral band clutches, expanding nitinol wire clutches, escapements, or other forms of overrunning clutches. Use of any such devices can still be within the spirit of the present system.

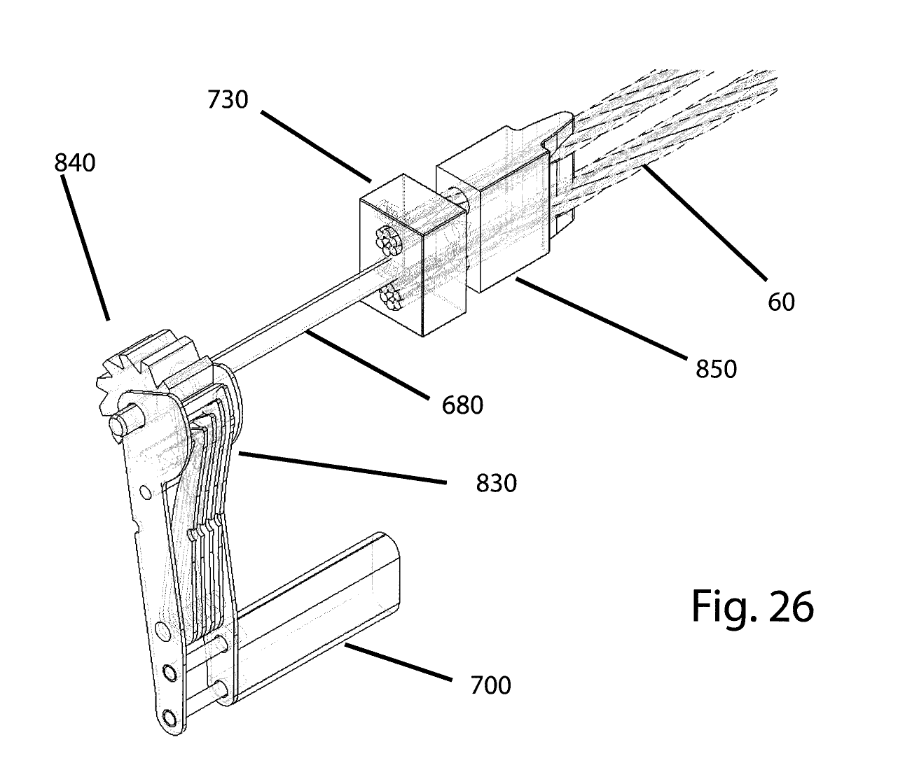

FIG. 26 shows one such variation. Here, multiple pawls 830 acting on a toothed ratchet wheel 840 are used to obtain fine pitch incremental rotation of shaft 680 in response to a small angular swing of arm 700 as previously described. Spring means not shown keep the various pawls lightly pressed against the ratchet wheel. The pawls are staggered and so arranged that the effective number of teeth on the ratchet wheel is the actual pitch multiplied by the number of pawls in use.

Not shown in FIG. 26 is the backstop prevention mechanism that can consist of a similar set of pawls pivoted on the actuator case and acting between the ratchet wheel 840 and the case.

FIG. 26 also illustrates an exemplary embodiment in which 730 is a nut threaded on shaft 680 (the threads are not shown in this Figure). The shaft rotates in bushings or bearings also not shown which prevent it from shifting axially within the housing or case 640.

FIG. 26 shows the present system as it can be configured for treating pectus excavatum with distractor links of the flattened bar type shown in FIG. 7. Base link 850 is rigidly attached to the actuator case 640 and serves as a reaction member against which the chain of distractor links is drawn as the cables are tensioned.

Rotation of threaded shaft 680 causes nut 730 to translate away from stationary component 850. In doing so it tensions cables 60 that are rigidly affixed to the nut 730 by set screws or other such devices.

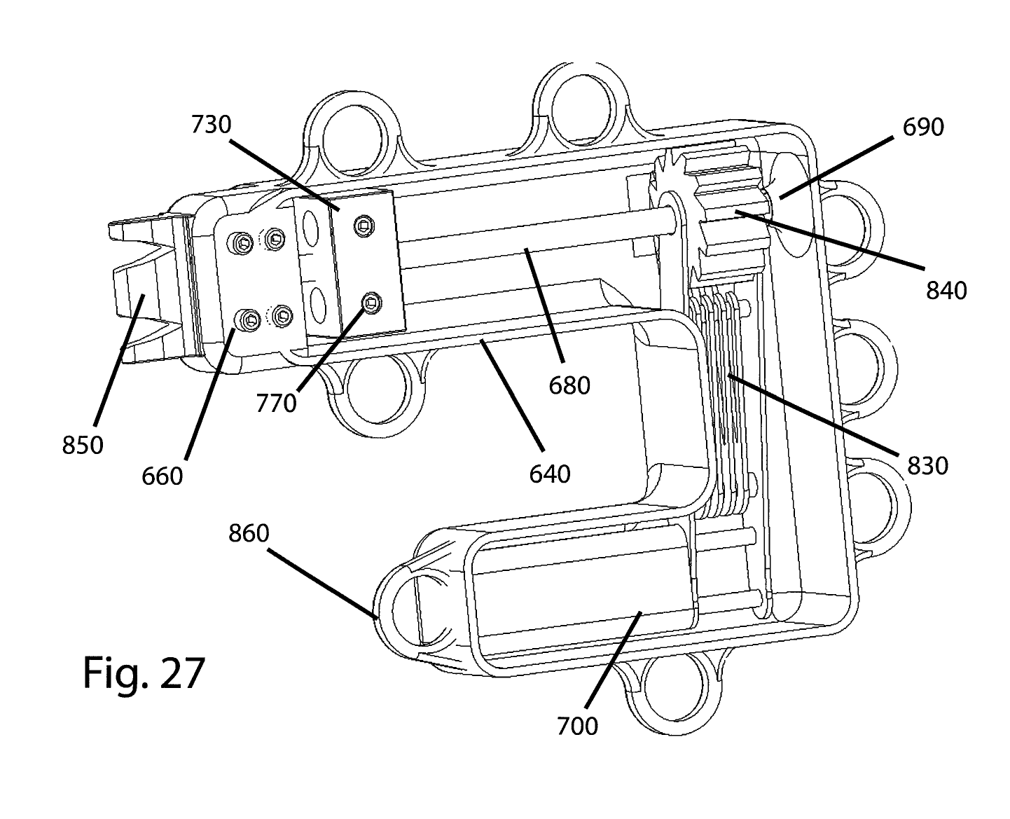

FIG. 27 shows an alternate cut-away view of FIG. 26. Here actuator case 640 is held fixed relative to the patient by suitable means such as stitch anchors 860. The reference base link 850 of the distractor mechanism is initially installed and positioned relative to the actuator case by means such as the setscrews 660. Using this means, the surgeon can pretension the cables as needed and take out initial slack in the distractor chain of links prior to closing the surgical incisions.

In FIG. 27, the nut 730 is shown with a substantially rectangular cross-section and slides in a mating prismatic slot in the case 640 so as to prevent rotation of the nut.

Relatively high tension can be achieved in the cables by using a fine pitch thread on shaft 680.

Shaft 680 rotates in one or more bearings or bushings such as at 690. The axial force due to the cable tension is taken up by a thrust bearing means acting between the rotating shaft and the case. For instance, said thrust bearing can be a rotating conical bushing or joint between shaft 680 and fixed link 850 attached to case 640.

FIG. 28 shows a form of the incremental actuator 10 that is activated by mechanical pressure on the two arms 870 and 880 of the wrap spring clutch 890. This does not require the electromagnet 30 or the electromechanical control system 40 but is purely mechanical. It is suited to use in situations such as pectus excavatum where the actuator can be mounted just beneath the skin and where the surgeon can actually press on the actuator from outside the body.

In this situation, a flexible diaphragm (not shown) can cover the wrap spring clutch portion of the case 640. Wrap spring clutch 890 is a loose fit on drum portion 900 of shaft 680.

Pressing simultaneously on arms 230 and 240 tightens the clutch coil 890 on the drum 260. Rocking one way or the other while doing so will incrementally rotate the shaft 680. In this way the surgeon can cause nut assembly 730 to advance or retract as desired along the screw threads 740 affixed to the shaft 680. A surface of the nut assembly 730 can slide against a guide surface such as 920 to keep the nut from rotating with respect to the case.

As described before, the shaft 680 is constrained to rotate on fixed bearings relative to the stationary members. In this case, a suitable socket at 910 can serve as such a rotating and thrust bearing device. Cables or other devices can be coupled to the actuator via means such as the nut assembly 730 and setscrews 770. These cables can then move the elements of the distractor 20 during the course of the treatment applied postoperatively.

Stitch anchors 860 or other attachment devices can be suitably disposed to aid in attaching the case 640 to the patient. A stationary base link member 850 can be fixed relative to the case to provide a reaction member to align and constrain the remainder of the distractor chain relative to the case as the nut assembly is progressively advanced during the course of treatment.

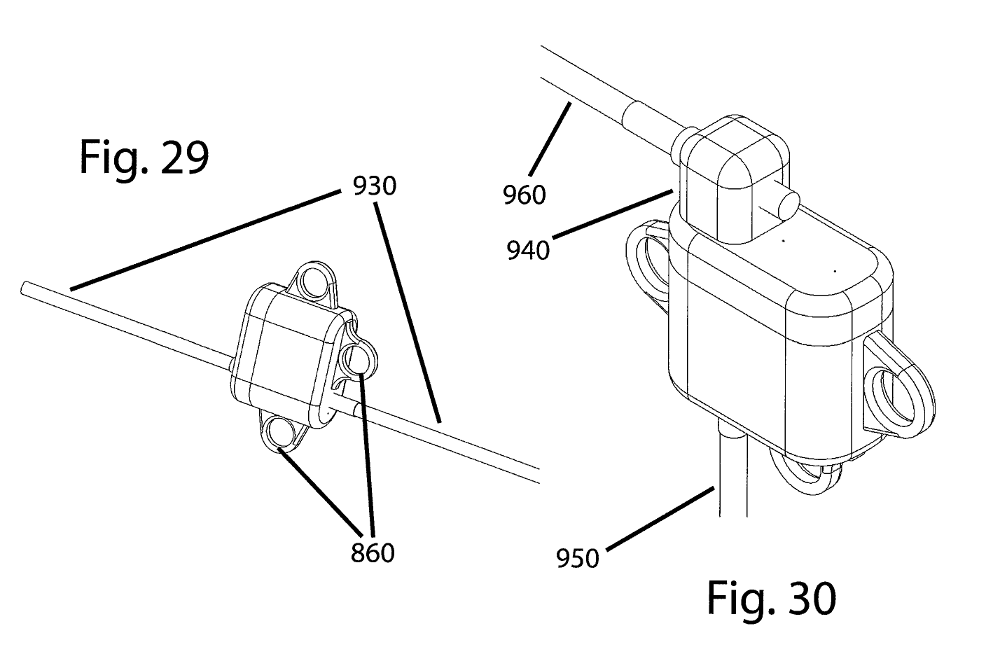

FIG. 29 shows how the incremental rotary actuator of the present system can be configured with double-ended shafts 930 if so desired for a particular surgical application. Each of these shafts can be used to drive distractor devices as previously described.

Other implanted medical devices suited to being driven by a periodically activated externally controlled rotary actuator can also be coupled to the actuator of the present system. Such devices can include specialized pumps or medicine release devices for example.

FIG. 29 also shows how attachment devices such as 860 can be added to the actuator to aid in suturing the actuator case to the patient or otherwise stabilizing it relative to the patient or the distractor mechanism.

FIG. 30 illustrates how a gearbox 940 can be employed in conjunction with the rotary actuator of the present system. Such a device can be used to change the speed, direction of rotation, or mechanical advantage or to obtain a rotary output on a secondary shaft 960 at an angle inclined in relation to the primary actuator shaft 950.

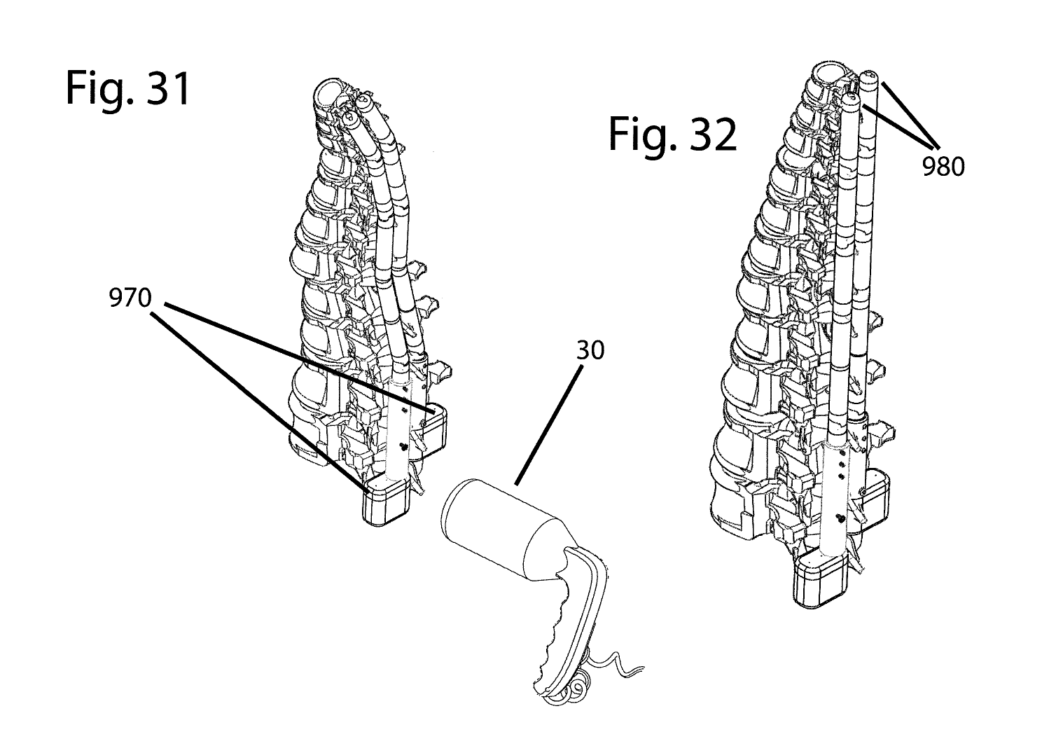

FIG. 31 shows how multiple distractors of the present system can be used, for example, in correcting scoliosis. With proper choice of end effectors, multiple distractors can be arranged to work cooperatively so as to give higher forces or better control of the distraction. The two distractor systems can need to have appropriate degrees-of-freedom and enough compliance so that each can be slightly adjusted with respect to the other without binding.

In this example, a pair of actuators is arranged at staggered locations 970. This spatial separation can allow them to be individually activated by the external transmitter 30. If the present system is used with other types of actuators then they can be individually addressed and controlled without being spatially separated.

FIG. 32 shows what this configuration of distractors can look like after treatment. It can be seen, for example at 980, that there can be provisions for some of the distractor links to slide or rotate in certain dimensions relative to their counterpart links on the second distractor and relative to the vertebrae as the correction took place. End effectors allowing such differential motion to occur are described as part of the present system.

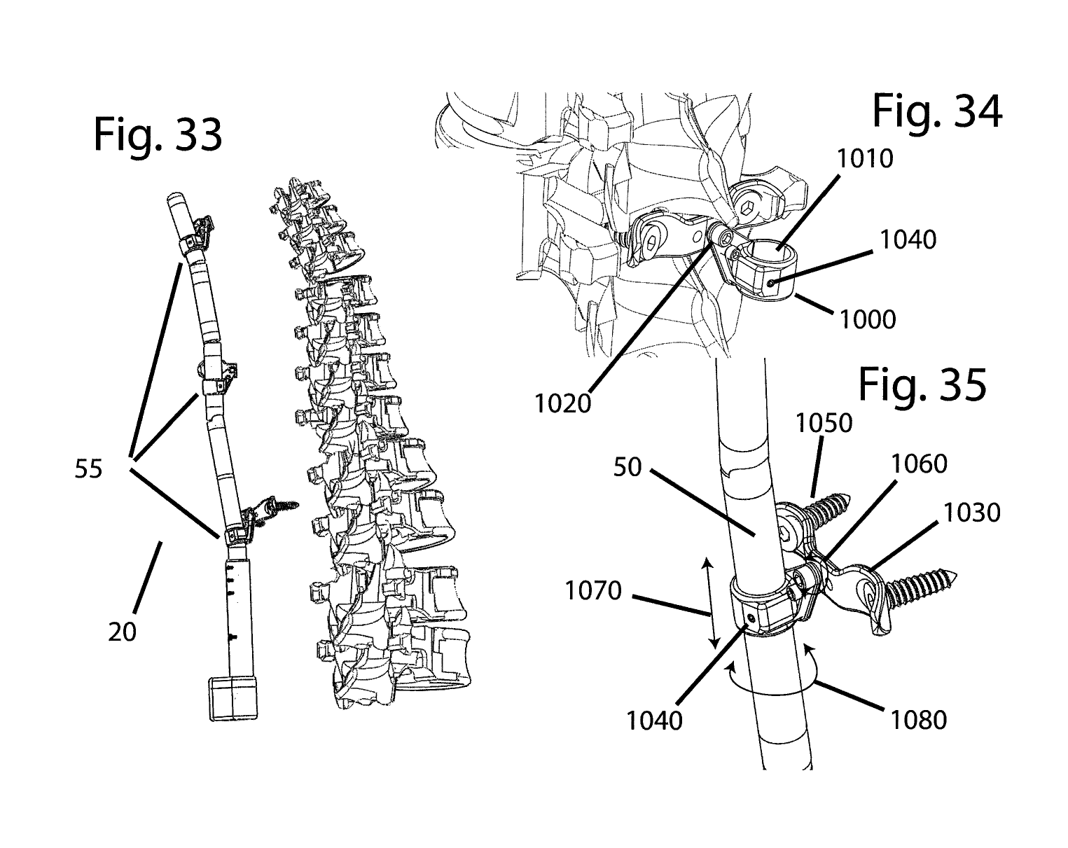

FIG. 33 illustrates some possible end effectors 55 of the present system. Many fixation devices, such as pedicle screws 1050, can also be adapted for use with this distraction system but a number of variations are described in the following figures. These new end effector variations of the present system are also adaptable for use with other distraction systems.

Conventional spinal fixation devices are designed to work with a static system of distraction rods rather than a hinged, changing distraction structure. The present system describes a dynamically reconfiguring jointed structure 20 where the slopes and positions of individual rod segments progressively change during the course of treatment.

“Dynamic” or semi-constrained couplings have been used in the past, but for the purpose of allowing the vertebrae to settle or move with respect to a stationary rod system, rather than in conjunction with a dynamically changing distractor 20 as described in the present system. The present system describes dynamic couplings and mechanical limit stops that not only meet those requirements but can additionally be configured to perform tasks unique to the requirements of the jointed link distractor 20 of the present system.

Individual links of distractor 20 must be able to preferentially bear on the tissues in certain directions and allow the tissues to shift with respect to the distractor links in certain other desired directions. End effector constraints needed at various points can also vary along the length of the distractor mechanism.

Certain distractor links can require semi-rigid anchoring to an anatomical structure such as a particular vertebra for example. Other links can require no coupling whatsoever to the anatomy, and others can, for example, require only side-to-side force transmission and need somewhat free motion in all other dimensions. This freedom can be needed to accommodate motion due to correction of the deformity, to allow growth of the patient, or to accommodate changes in the geometry of the distractor 20 itself. Constructions described in the present system can satisfy each of these varied requirements.

End effectors of the present system can be rigidly coupled to the jointed distractor links or coupled by slack motion couplings utilizing cam slots, cylindric joints, or pivots with controlled clearances. FIGS. 34 through 40 show such end effector variations of the present system particularly adapted to coupling links of the jointed distractor mechanism to individual vertebrae with selectable degrees of freedom or constraint.

In FIG. 34, part 1000 comprises a member that can be mounted to a particular distractor link 50, either by bolting to the link or by wrapping around the link as shown. Part 1000 can be rigidly fastened to the distractor link 50 or it can be coupled to the link by a bearing allowing motion in one or more directions. Bore 1010 in part 1000 can be a tight fit rigidly holding 1000 to link 50 or it can be oversize to form a bearing allowing rotational motion such as 1080 or axial translational motion such as 1070 with respect to 50. A setscrew such as 1040 can be used to lock part 1000 in position with respect to 50 and suitable stops such as collar 1090 held in place by a setscrew 1100 can be used to limit the range of motion allowed.

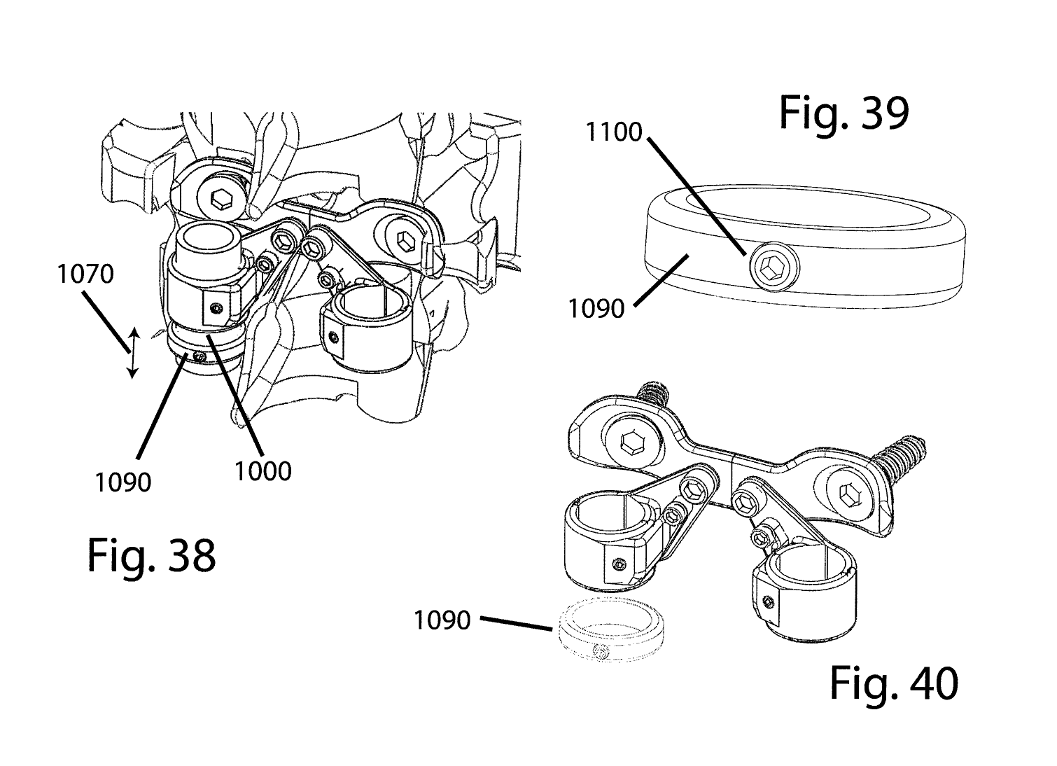

As illustrated in FIG. 38, limit stops can be arranged to allow portions of the distraction system to move freely in certain directions such as 1070 when treatment begins. In this example, the connected end effectors only begins applying distraction force to the connected anatomical structures when the stop 1090 hits its limits and begins pressing upwards on the bottom of 1000. This aspect of the present system can minimize initial loads on the distractor system and allow the distractor 20 to selectively engage and exert certain forces at a planned later point in the progressive treatment protocol when it is desirable to do so, either for medical or for structural mechanical reasons. For instance, as the joints of the distractor near collinearity, (a “toggled” position) the distractor mechanism 20 will be able to generate higher axial forces than when there is more freedom for angular articulation in the joints.

FIG. 35 shows how part 1000 can be mounted to a connecting bracket or plate 1030 by means of a bolt 1020 passing through a hole 1160 or other suitable mounting means. Fixation bracket 1030 can in turn be mounted to a vertebra by pedicle screws 1050 or other suitable devices.

Attachment 1020 can either provide a rigid coupling or one or more degrees of freedom. For instance, if bolt 1020 is slack it can serve as a hinge joint allowing rotation about its axis as shown by 1060. The coupling between the bolt 1020 and the fixation plate 1030 can also be designed with more freedom, allowing it to function as a ball joint. Further, 1160 can be designed as an elongated slotted coupling as shown by 1110 in FIG. 37. As shown in this illustrative example, 1020 moving in the slot can provide constraint in the axial direction but limited freedom of motion from side-to-side. When the bolt reached the end of the slot 1110 it can begin exerting sideways force in the side-to-side direction 1120.

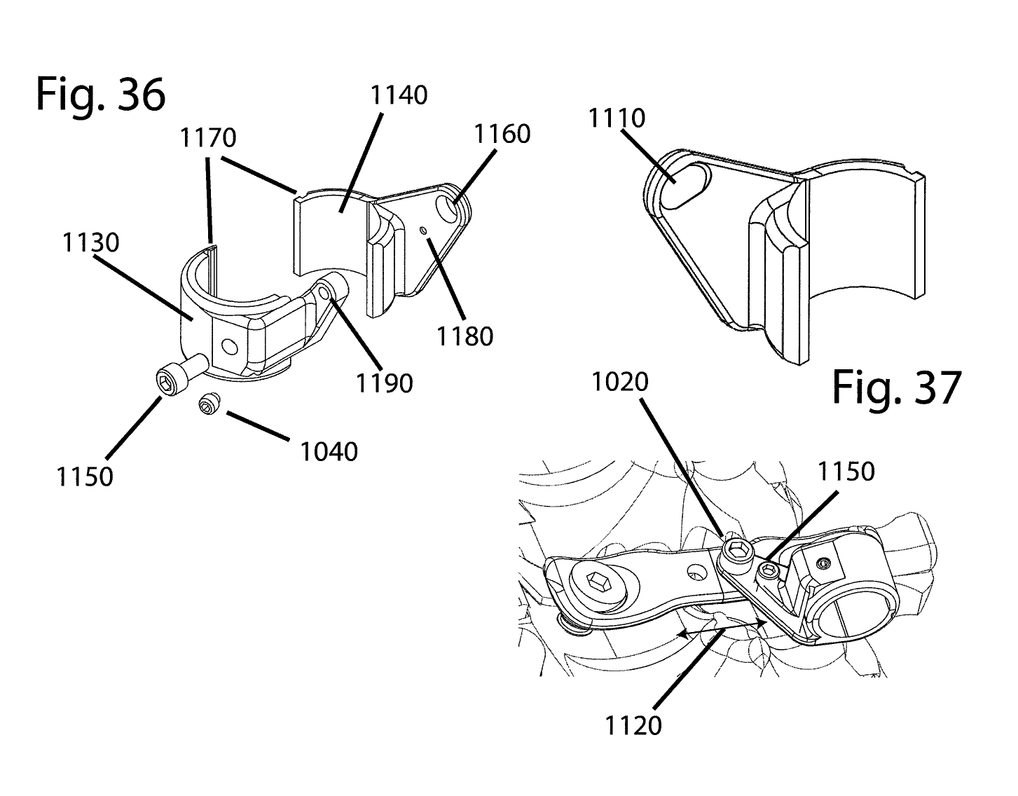

FIG. 36 shows an exemplary embodiment of this end effector in which part 1000 comprises two components 1130 and 1140. Cover member 1130 and inner member 1140 snap together, keyed by the mating edges as at 1170. They are then locked in position by bolt 1150 passing through hole 1190 in cover member 1130 and threaded into mating hole 1180 in the inner member 1140.

This construction clears up the surgical field and simplifies the assembly of the distractor system 20 during surgery. Parts of the distractor system lying near the spine can be attached to the vertebrae without interference from the distractor links 50. Then, the distractor links 50 can be positioned, and finally the cover member 1130 can be bolted in place.

FIGS. 38 and 40 illustrate how the present system can be adapted to work with a pair of distractor links on either side of the spine.

Further control of the distractor system 20 of the present system can be achieved by incorporating flexural elements such as springs into the links, joints, or end effectors of the present system. Such compliant elements can also be arranged to provide protection against accidental overloads, say due to an unforeseen motion, impact, or load on the tissues.

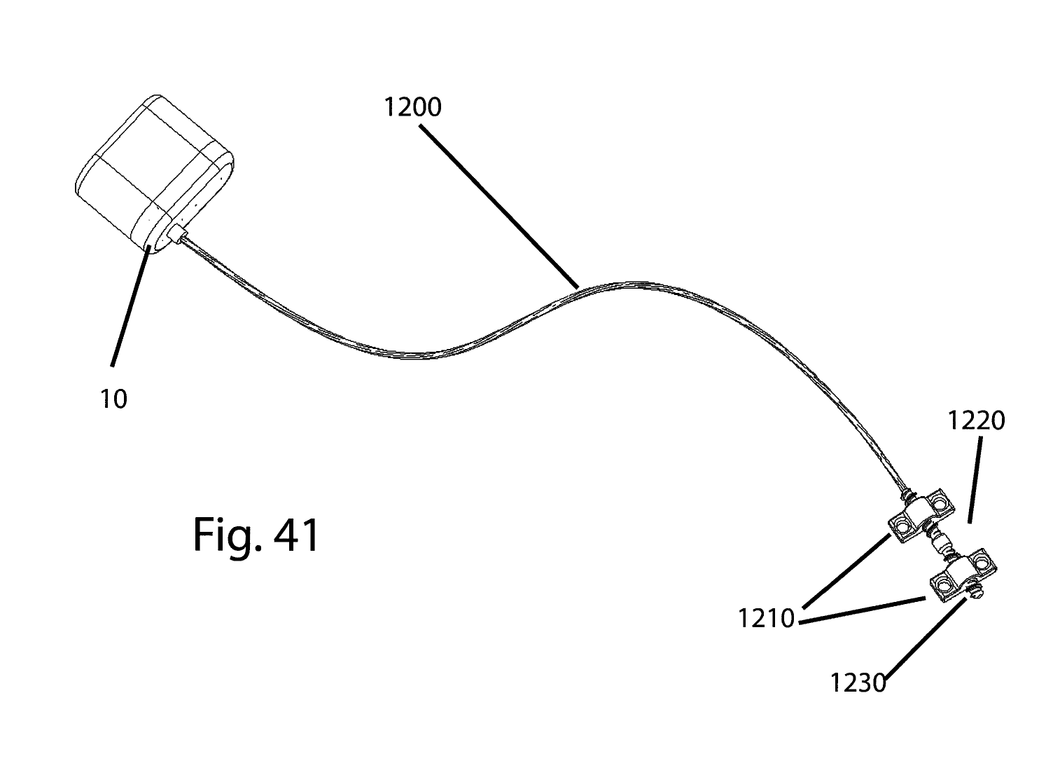

FIGS. 41 through 46 show the present system with the incremental actuator 10 of the present system remotely coupled to an implanted end effector distraction device 1220 means by use of flexible shafting 1200 or a variety of other flexible coupling devices. In FIG. 41, the remote end effector 1220 comprises a turnbuckle assembly. Shaft 1230 of the turnbuckle end effector mechanism 1220 has portions with left and right hand screw threads 1240. These engage matching left and right hand threaded end effector nut members 1210 that can be shaped as needed and can be sutured, screwed, or otherwise attached to the bones or other tissues being distracted using suitable attachment features like the holes 1250.

For instance, in one possible application of use of this system in craniofacial surgery, this system can be adapted for use in distracting skull sutures in an infant. Actuator 10 can be remotely located beneath the skin.

The flexible shaft 1200 can be designed to have torsional stiffness in the direction of the actuator rotation.

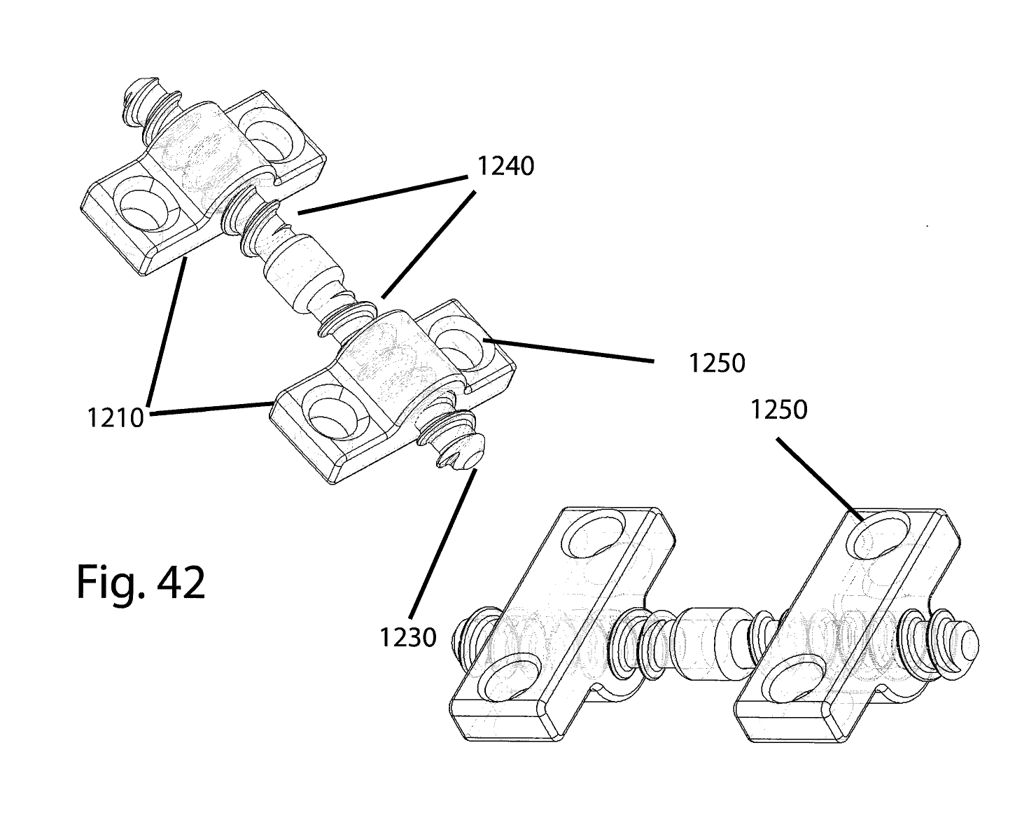

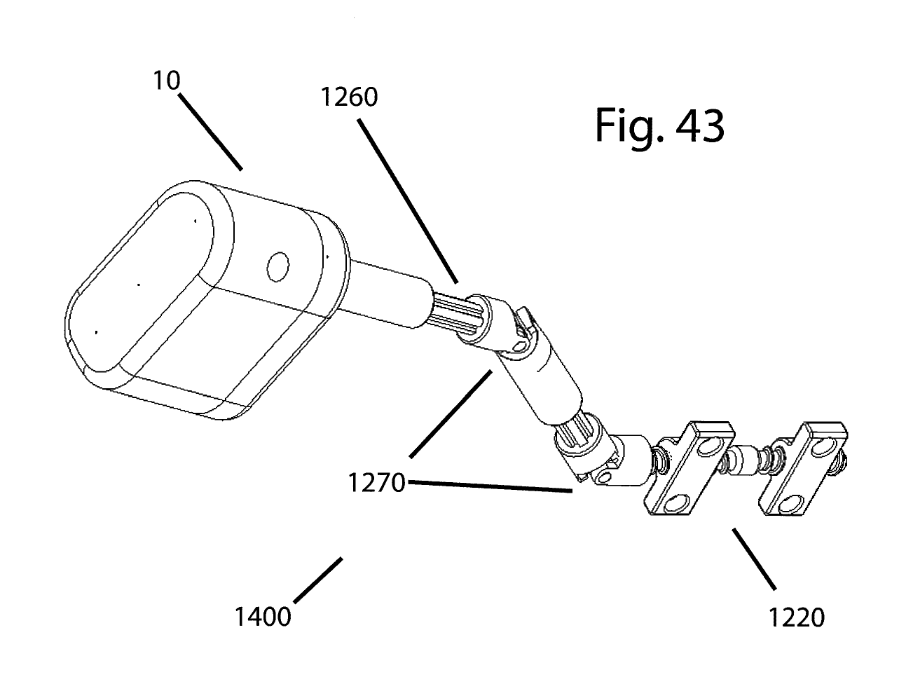

FIG. 42 shows an illustrative turnbuckle-style distractor mechanism that can be used in conjunction with an implanted rotary actuator such as 10. Clockwise and counterclockwise screw threads or screws of different pitches can be employed to cause the end effectors 1210 to move together or apart as needed, even though the input rotation is unidirectional. High axial forces can be obtained by utilizing the differential action of such a mechanism. If desired, a rotary bearing can be employed in place of one of the threaded connections since a rotary bearing is kinematically equivalent to a screw thread of zero pitch.

FIG. 43 illustrates an apparatus in which a flexible drive train 1400 comprising a chain of prismatic 1260 and universal joints 1270 is used to couple the actuator 10 to the remote turnbuckle end effector system 1220. In this way the actuator can be located in a convenient preferred location remote from the region in which the distraction is required, such as where there is more available space within the patient or where the magnetic pulses can be better transmitted to the actuator.

It will be appreciated from FIGS. 41 and 43 that within the spirit of this system the flexible drive train 1400 can comprise shafts, bellows, flexible couplings, universal joints, and similar devices as needed to transmit the rotary motion from the actuator 10 to the actual distractor end effector mechanism 1220.

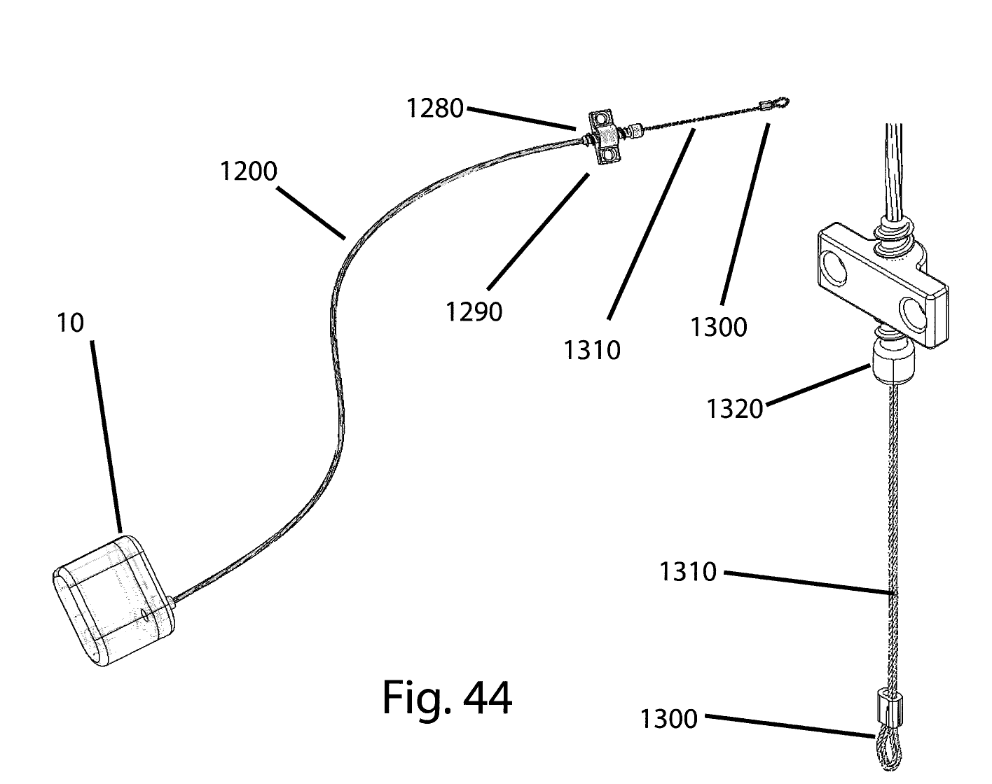

FIG. 44 shows the present system with an end effector distractor which comprises a tensioned cable 1310. As illustrated, a flexible shaft 1200 couples the rotary actuator 10 to a threaded member 1280. Threaded nut component 1290 comprises one half of the end effector system and is screwed or sutured in position as described earlier in connection with the turnbuckle.

Rotation of said threaded member 1280 in the matching threaded nut component 1290 causes the threaded member 1280 to move axially with respect to the nut 1290. This axial motion can be used to tighten or loosen a cable 1310. Said cable is attached between nut end effector member 1290 and end effector 1300 affixed to the tissues to be distracted with respect to those affixed to 1290. The loop 1300 just shows one of many ways the cable can be attached to the tissues or bones at the distal end of the cable 1310. If desired there can be an intermediate rotary bearing 1320 so as to avoid producing a twist in the cable 1310.

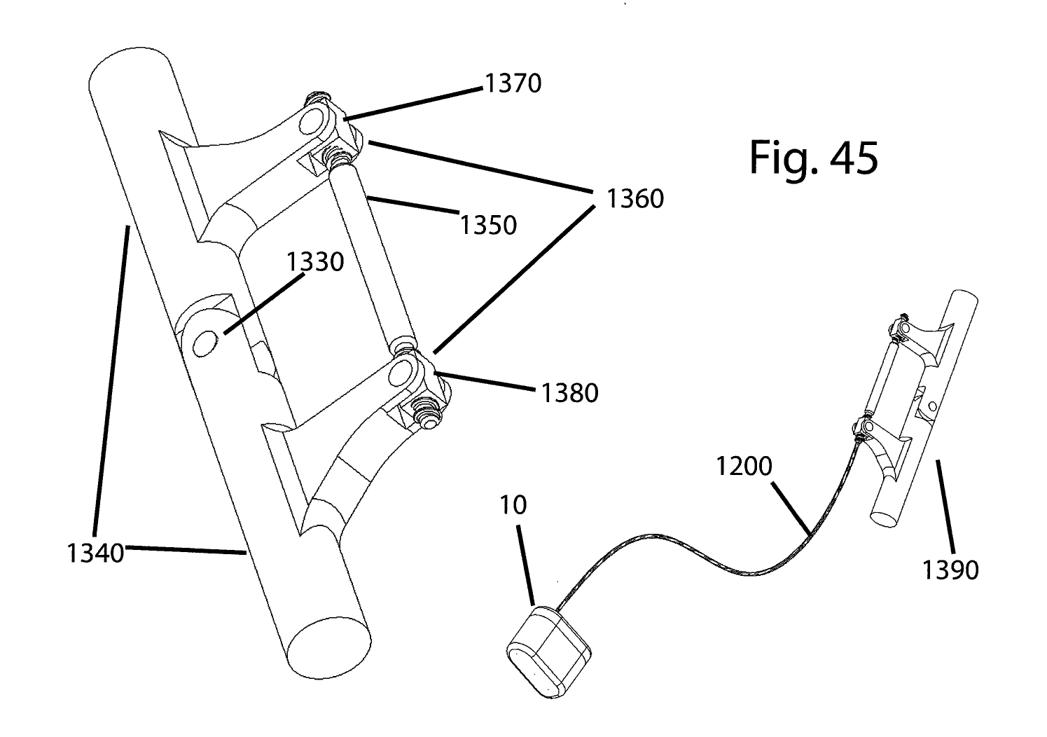

FIG. 45 shows another style of articulated distractor 1390 that can be driven by the rotary actuator 10 of the present system using a flexible drive shaft 1200 or other flexible drive train coupling such as 1400 taught in this patent.

Shaft member 1350 has clockwise and counterclockwise threaded distal portions mated with threads in pivoted block members 1370 and 1380. Rotation of shaft member 1350 effectively pushes apart or pulls together points 1360 thereby causing the joint between links 1340 to hinge about point 1330. Motion of the distractor links 1340 is then coupled to the tissues by suitable end effectors or shaped surfaces built into or attached to members 1340.

It will be appreciated that other differential screw configurations and linkage geometries can be employed to similarly achieve articulation between links 1340 of a distractor of the present system.

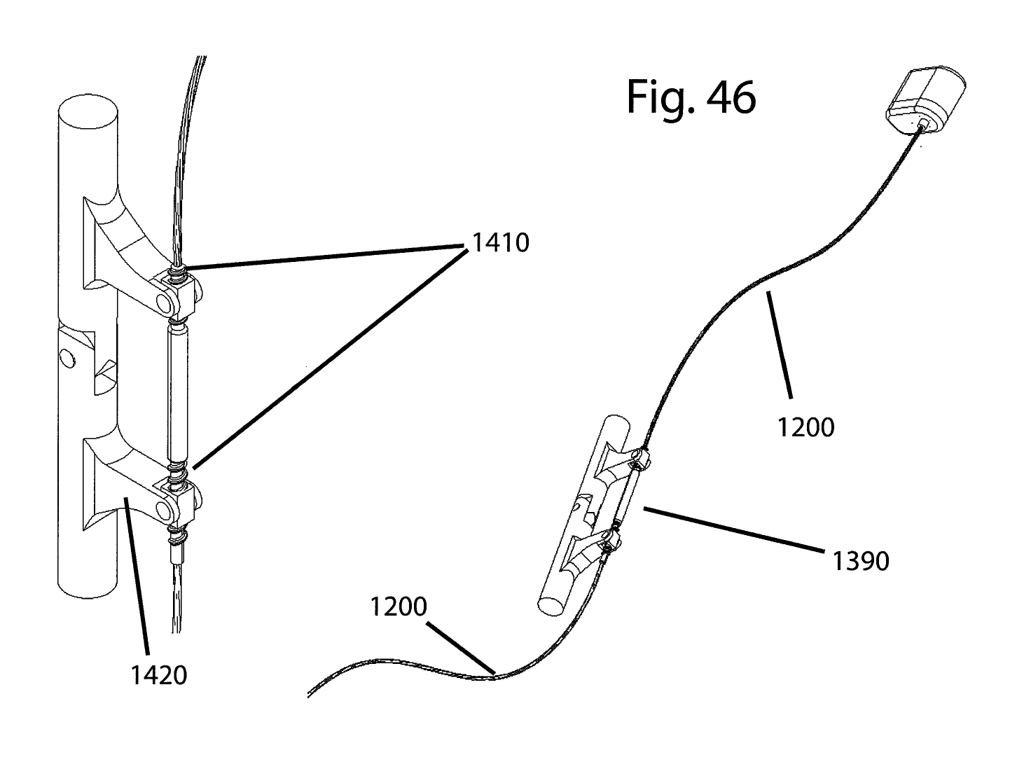

FIG. 46 shows how multiple articulated distractor joints 1390 can be chained together and serially driven by a single rotary actuator 10. By using different choices of screw threads on the different joints 1410 or by using different lever arms 1420 one can obtain differential action along the length of the distractor assembly. Joints can be designed to rotate in different planes, in opposite directions, and by different amounts as needed. High mechanical advantages can be attained by having rotation of the actuator driving fine pitch threaded connections to the output end effector members or by having longer effective lever arms 1420. The link construction shown where the two links 1340 are co-linear is merely illustrative. The links can be made like bell cranks with the joint halves at right angles to one another or at another desired angle, depending on the anatomical needs of the particular application.

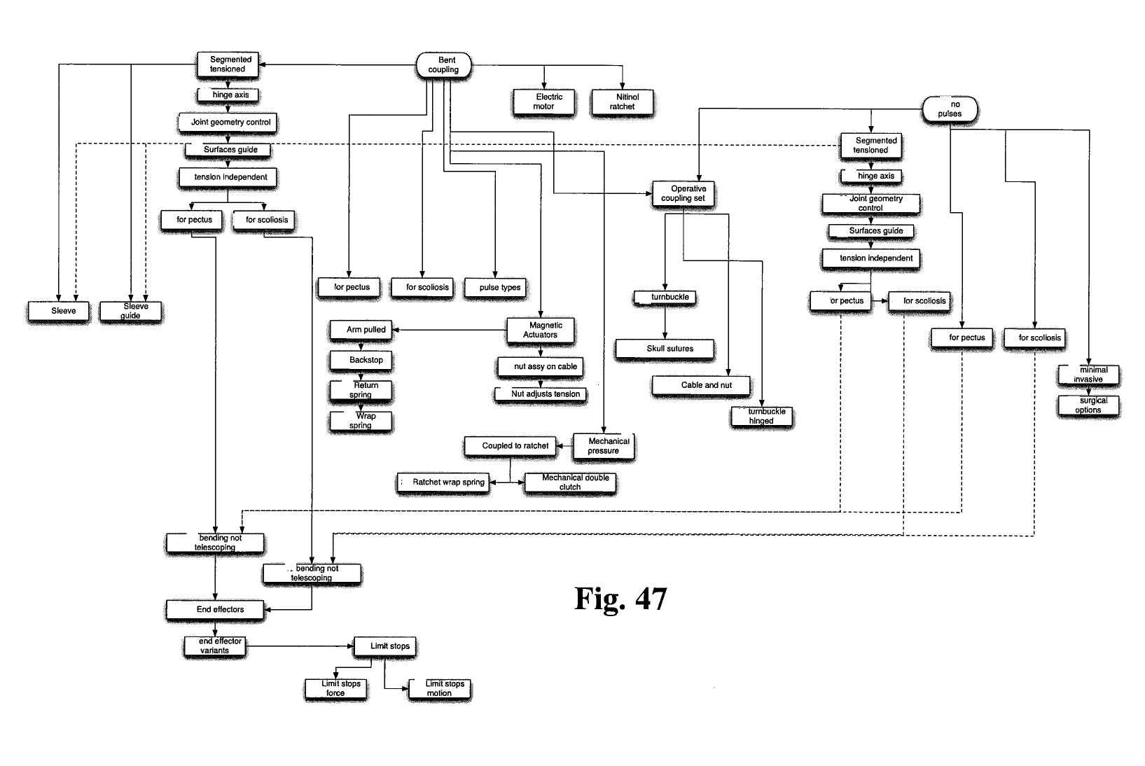

FIG. 47 shows a chart illustrating possible variations of the implantable surgical distraction apparatus, including variations of components of the apparatus, variations of the features, variations of applications of the apparatus, and combinations of the different variations. These include bent coupling, segmented tensioned, hinge axis, joint geometry control, surfaces glide, tension independent, for pectus, for scoliosis, bending not telescoping, end effectors, end effector variants, limit stops, limit stops force, limit stops motion, sleeves, sleeve guides, pulse types, electric motor, nitinol ratchet, magnetic actuators, arm pulled, backstop, return spring, wrap spring, nut assembly on the cable, nut adjusts tension, mechanical pressure, coupled to ratchet, ratchet wrap spring, mechanical double clutch, operative coupling set, turnbuckle, skull sutures, cable and nut, turnbuckle hinged, no pulses, minimally invasive surgical tools, and surgical options.

We claim:

1. A surgical distraction apparatus comprising:
a tissue distraction device adapted for implantation, the tissue distraction device comprising a plurality of distraction members;
one or more actuators adapted for implantation and operatively coupled to the tissue distraction device, wherein the one or more actuators are configured to drive the tissue distraction device and adjust the alignment of the plurality of distraction members; and
an activator adapted to deliver one or more pulses to at least one actuator in the one or more actuators.

2. The apparatus of claim 1, wherein the plurality of distraction members are coupled by one or more cables and are arranged so as to provide a plurality of distraction forces in response to a tension force in the one or more cables and wherein at least one distraction force in the plurality of distraction forces is transverse to the tension force in the one or more cables.

3. The apparatus of claim 2, wherein two or more distraction members in the plurality of distraction members are configured to rotate with respect to one another as at least one cable of the one or more cables is tightened.

4. The apparatus of claim 3, wherein rotation of the two or more distraction members with respect to one another is determined at least in part by the geometry of joints between the two or more distraction members.

5. The apparatus of claim 4, wherein a first surface on a first distraction member in the two or more distraction members engages a second surface on a second distraction member in the two or more distraction members to guide the first distraction member and the second distraction member into alignment as the at least one cable is tightened.

6. The apparatus of claim 5, wherein the bending stiffness of a compound distraction member comprising the first distraction member and the second distraction member is independent of tension in the at least one cable when the first distraction member and the second distraction member are in complete alignment.

7. The apparatus of claim 6, wherein one or more distraction members in the plurality of distraction members are configured for tunneling beneath a sternum and wherein tightening the one or more cables causes mating surfaces on the one or more distraction members to engage one another, stiffening the one or more distraction members and creating one of an outward corrective force on the sternum for a patient with pectus excavatum or an inward corrective force on the sternum for a patient with pectus carinatum.

8. The apparatus of claim 6, wherein one or more distraction members in the plurality of distraction members are configured to apply a force to one or more vertebrae of a spine and wherein tightening the one or more cables causes mating surfaces on the one or more distraction members to engage one another, stiffening the one or more distraction members and creating a corrective force on the spine for a patient with scoliosis.

9. The apparatus of claim 2, wherein the tissue distraction device is adapted to correct one or more of pectus excavatum and pectus carinatum.

10. The apparatus of claim 2, wherein the tissue distraction device is adapted to correct scoliosis.

11. The apparatus of claim 1, wherein the one or more pulses comprise at least one of magnetic pulses, electromagnetic pulses, or mechanical pulses.

12. The apparatus of claim 1, wherein the activator comprises an electromagnetic activator and wherein the one or more actuators are magnetically driven and include a ratchet mechanism comprising a hinged arm member configured to incrementally rotate a shaft when the electromagnetic activator is energized and to return to a rest position when the electromagnetic activator is de-energized.

13. The apparatus of claim 12, wherein the hinged arm member rotates through an angular displacement about its hinge axis when a portion of the hinged arm member which is offset from the hinge axis is attracted by the electromagnetic activator in an energized state and wherein the hinged arm member returns to a rest position when the electromagnetic activator is de-energized.

14. The apparatus of claim 13, wherein the shaft rotates in a first direction and wherein the one or more actuators include a second ratchet mechanism configured to prevent the shaft from rotating in a second direction opposite the first direction when the hinged arm member returns to the rest position.

15. The apparatus of claim 14, wherein the one or more actuators include a return spring configured to return the hinged arm member to its rest position when the electromagnetic activator is de-energized.

16. The apparatus of claim 15, wherein at least one of the first ratchet mechanism and the second ratchet mechanism comprise a wrap spring clutch.

17. The apparatus of claim 12, wherein the plurality of distraction members are coupled by one or more cables and wherein the shaft is mounted on a bearing member and comprises a threaded portion operatively coupled to one or more nuts which are mechanically coupled to at least one cable in the one or more cables.

18. The apparatus of claim 17, wherein the axial motion of the one or more nuts adjusts tension in the at least one cable.

19. The apparatus of claim 1, wherein the one or more pulses cause an alternating increase and decrease of mechanical pressure on one or more external points of a patient, the one or more external points being proximal to the at least one actuator in the one or more actuators.

20. The apparatus of claim 19, wherein the alternating increase and decrease of mechanical pressure is effected through tissues belonging to the patient by mechanically driving a ratchet mechanism within the at least one actuator when the at least one actuator is implanted in the patient.

21. The apparatus of claim 20, wherein the ratchet mechanism comprises a wrap spring clutch.

22. The apparatus of claim 21, wherein the wrap spring clutch comprises:
a coil spring having a first end and a second end; and
a shaft positioned coaxially with the coil spring and adapted to rotate around a common axis;
wherein the coil spring is adapted to transmit driving torque to the shaft when differential force is applied to the first end and the second end to thereby rotate the shaft.

23. The apparatus of claim 1, wherein at least one of the one or more actuators includes an electric motor.

24. The apparatus of claim 1, wherein at least one of the one or more actuators includes a nitinol ratchet.

25. A surgical distraction apparatus comprising:
a tissue distraction device adapted for implantation, the tissue distraction device comprising a plurality of distraction members; and
one or more actuators adapted for implantation and operatively coupled to the tissue distraction device, wherein the one or more actuators are configured to drive the tissue distraction device and adjust the alignment of the plurality of distraction members;
wherein activation of at least one actuator in the one or more actuators causes a three dimensional displacement of least one distraction member in the plurality of distraction members.

26. The apparatus of claim 25, wherein the plurality of distraction members are coupled by one or more cables and are arranged so as to provide a plurality of distraction forces in response to a tension force in the one or more cables and wherein at least one distraction force in the plurality of distraction forces is transverse to the tension force in the one or more cables.

27. The apparatus of claim 26, wherein two or more distraction members in the plurality of distraction members are configured to rotate with respect to one another as at least one cable of the one or more cables is tightened.

28. The apparatus of claim 27, wherein rotation of the two or more distraction members with respect to one another is determined at least in part by the geometry of joints between the two or more distraction members.

29. The apparatus of claim 28, wherein a first surface on a first distraction member in the two or more distraction members engages a second surface on a second distraction member in the two or more distraction members to guide the first distraction member and the second distraction member into alignment as the at least one cable is tightened.

30. The apparatus of claim 29, wherein the bending stiffness of a compound distraction member comprising the first distraction member and the second distraction member is independent of tension in the at least one cable when the first distraction member and the second distraction member are in complete alignment.

31. The apparatus of claim 30, wherein one or more distraction members in the plurality of distraction members are configured for tunneling beneath a sternum and wherein tightening the one or more cables causes mating surfaces on the one or more distraction members to engage one another, stiffening the one or more distraction members and creating one of an outward corrective force on the sternum for a patient with pectus excavatum or an inward corrective force on the sternum for a patient with pectus carinatum.

32. The apparatus of claim 30, wherein one or more distraction members in the plurality of distraction members are configured to apply a force to one or more vertebrae of a spine and wherein tightening the one or more cables causes mating surfaces on the one or more distraction members to engage one another, stiffening the one or more distraction members and creating a corrective force on the spine for a patient with scoliosis.

33. The apparatus of claim 26, wherein the tissue distraction device is adapted to correct one or more of pectus excavatum and pectus carinatum.

34. The apparatus of claim 26, wherein the tissue distraction device is adapted to correct scoliosis.

35. The apparatus of claim 25, wherein the at least one actuator in the one or more actuators is configured for adjustment using a minimally invasive surgical tool when implanted in a patient.

36. The apparatus of claim 35 wherein the minimally invasive surgical tool comprises at least one of a surgical screwdriver, a surgical wrench, setscrews, surgical pliers, and a manipulable component of the at least one actuator.

37. The apparatus of claim 25, wherein the one or more actuators are operatively coupled to the tissue distraction device using at least one of a shaft, a flexible shaft, a bellows, a flexible coupling, a universal joint, and a telescoping prismatic joint.

38. The apparatus of claim 37, wherein surgical distraction apparatus further comprises:
a threaded shaft member adapted to rotate;
a nut member coupled to the threaded shaft so as to allow axial motion of the threaded shaft with respect to the nut member when the shaft rotates;
a tissue attachment enabler comprising at least one of suture holes and bone screw holes disposed on the nut member, the tissue attachment enabler adapted to receive an attachment member which attaches the nut member to a first set of tissues;
a cable providing a tension connection between the threaded shaft member and a second set of tissues; and
a rotating bearing adapted to prevent twisting of the tension cable

39. The apparatus of claim 37, wherein surgical distraction apparatus further comprises:
a shaft member adapted to rotate;
a first nut member and a second nut member coupled to the shaft and configured to move differentially along the axis of the shaft when the shaft rotates;
a first tissue attachment enabler comprising at least one of suture holes and bone screw holes disposed on the first nut member, the first tissue attachment enabler adapted to receive a first attachment member which attaches the first nut member to a first set of tissues; and
a second tissue attachment enabler comprising at least one of suture holes and bone screw holes disposed on the second nut member, the second tissue attachment enabler adapted to receive a second attachment member which attaches the second nut member to a second set of tissues;
wherein differential motion of the first nut and the second nut provides distraction forces between the first set of tissues and the second set of tissues.

40. The apparatus of claim 37, wherein surgical distraction apparatus further comprises:
a shaft member adapted to rotate;
a first nut member and a second nut member coupled to the shaft and configured to move differentially along the axis of the shaft when the shaft rotates;
a first hinge coupling a first distraction member in the plurality of distraction members with the first nut member;
a second hinge coupling a second distraction member in the plurality of distraction members with the second nut member;
a third hinge coupling the first distraction member with the second distraction member, wherein the third hinge axis is not collinear with the shaft member;
a first attachment member which attaches the first nut member to a first set of tissues; and
a second attachment member which attaches the second nut member to a second set of tissues;
wherein differential motion of the first nut and the second nut provides distraction forces between the first set of tissues and the second set of tissues.

41. The apparatus of claim 33, wherein the at least one distraction force is generated by a relative bending or straightening of joints between at least two distraction members in the plurality of distraction members.

42. The apparatus of claim 34, wherein the at least one distraction force is generated by a relative bending or straightening of joints between at least two distraction members in the plurality of distraction members.

43. The apparatus of claim 39, wherein the tissue distraction device further comprises a turnbuckle apparatus adapted to the distraction of skull sutures.

44. The apparatus of claim 42, wherein the tissue distraction device further comprises one or more end effectors that act between one or more distraction members in the plurality of distraction members and the tissues being distracted.
one or more actuators operatively coupled to the tissue distraction device

45. The apparatus of claim 42, wherein the one or more end effectors comprise:
a first member that mounts to the anatomical tissues using at least one of pedicle screws, sutures, and plates; and
a second member mounted to the one or distraction members and operatively coupled to the first member so as to transmit distraction forces between the first member and the second members;
wherein the one or more actuators are operatively coupled to the tissue distraction device using one or more of rigid connections, ball joint connections, hinge connections, cylindrical connections, prismatic connections, slotted connections, cam connections, movable connections with stops limiting range of motion, adjustable lockable connections, setscrew devices, collet devices, and motion limiting collars.

46. The apparatus of claim 45, wherein the one or more end effectors incorporate limit stops configured to allow limited relative motion between the first member and the second member prior to the limit stops coming into contact.

47. The apparatus of claim 46, wherein the limit stops are configured to transmit force between the first member and the second member in the direction of their common normal at their point of contact once the limit stops come into contact.

48. The apparatus of claim 46, wherein the limit stops are configured to prevent the first member and the second member from moving towards one another along the line of action of their common normal at their point of contact once the limit stops come into contact.

49. The apparatus of claim 26, further comprising a flexible sleeve configured to enclose one or more joints between one or more distraction members in the plurality of distraction members and prevent tissues from entering the one or more joints.

50. The apparatus of claim 26, further comprising a flexible sleeve with markings configured to aid a surgeon in inserting one or more distraction members in the plurality of distraction members.
